(19) United States
(12) Patent Application Publication
(10) Pub. No.: US 2017/0176470 A1
(43) Pub. Date: Jun. 22, 2017

(54) DETECTION OF CARBOXYALKYLPYRROLE OR PENTYLPYRROLE ETHANOLAMINE PHOSPHOLIPIDS

(71) Applicant: CASE WESTERN RESERVE UNIVERSITY, Cleveland, OH (US)

(72) Inventor: Robert G. Salomon, Cleveland, OH (US)

(21) Appl. No.: 15/345,165

(22) Filed: Nov. 7, 2016

Related U.S. Application Data
(60) Provisional application No. 62/252,236, filed on Nov. 6, 2015.
(60) Provisional application No. 62/353,096, filed on Jun. 22, 2016.

(51) Int. Cl. G01N 33/92; G06F 19/00; C12Q 1/44

(52) U.S. Cl. CPC G01N 33/92; C12Q 1/44; C12Y 301/04004; G06F 19/3431; G01N 2800/16; G01N 2800/50

(57) ABSTRACT

A method of detecting carboxyalkylpyrrole ethanolamine phospholipids (CAP-EPs) or pentylpyrrole ethanolamine phospholipids (CAP-EPs) in a bodily sample from a subject includes obtaining a bodily sample from a subject suspected of including carboxyalkylpyrrole ethanolamine phospholipids (CAP-EPs) or pentylpyrrole ethanolamine phospholipids (PP-EPs) extracting carboxyalkylpyrrole ethanolamine phospholipids or pentylpyrrole ethanolamine phospholipids from the bodily sample; hydrolyzing carboxyalkylpyrrole or pentylpyrrole ethanolamine phospholipids from the extracted with a phospholipase D to form carboxyalkylpyrrole ethanolamine (CAP-ETN) and pentylpyrrole ethanolamine (PP-ETN) derivatives; and determining the amount of carboxyalkylpyrrole ethanolamine (CAP-ETN) and pentylpyrrole ethanolamine (PP-ETN) by mass spectrometry.

### RELATED APPLICATION

This application claims priority from U.S. Provisional Application Nos. 62/252,236, filed Nov. 6, 2015 and 62/353,096 filed Jun. 22, 2016, the subject matter of which are incorporated herein by reference in their entirety.

### GOVERNMENT FUNDING

This invention was made with government support under Grant No. GM021249, EY016813 NEI awarded by The National Institutes of Health. The United States government has certain rights in the invention.

### BACKGROUND

Macular degeneration is the clinical term used to describe those diseases that are characterized by a breakdown of the macula, the small portion of the retina responsible for central vision. Juvenile macular degeneration, also referred to as early onset macular degeneration occurs early in life, such as for example in the second and third decade, while age-related macular degeneration (AMD) occurs later in life, typically in the fifth decade and later. AMD constitutes a major health problem for individuals over 55 years of age in the industrialized world. In the USA alone between 6 and 10 million senior adults are legally blind from AMD.

2-ω-Carboxyethylpyrrole (CEP)-protein derivatives are formed through post-translational modification of the ε-amino groups of protein lysyl residues by 4-hydroxy-7-oxo-hept-5-eonyl phospholipids (HOHA-PLs), which are uniquely generated from oxidation of docosahexaenoate containing phospholipids (DHAPLs). Using anti-CEP antibodies raised against CEP protein, CEP immunoreactivity was first detected in vivo in photoreceptor rod outer segments that are DHA-rich tissues and in retinal pigmented epithelium that endocytose oxidatively damaged rod outer segment tips. CEPs are found in human Bruch's membrane/retinal pigmented epithelium/choroid tissues and extracellular deposits termed drusen which are hallmarks of AMD.

It is desirable to have diagnostic methods for determining if an individual has a predisposition for developing age-related macular degeneration and other diseases which involve oxidative damage to tissues from oxidation of DHA-containing lipids.

### SUMMARY

Embodiments described herein relate to methods for the detection and measurement of carboxyalkylpyrrole ethanolamine phospholipids (CAP-EPs) and/or pentylpyrrole ethanolamine phospholipids (PP-EPs) or to their use in the clinical assessment of age-related macular degeneration (AMD) risk, CAP-associated tumor progression, and sickle cell disease, and for monitoring the efficacy of associated therapeutic interventions.

The methods can includes obtaining a bodily sample, such as a blood, plasma, or sera sample, from a subject, such as a subject having or at risk of AMD, cancer, or sickle cell disease. Carboxyalkylpyrrole ethanolamine phospholipids (CAP-EPs) and pentylpyrrole ethanolamine phospholipids (PP-EPs) are then extracted from the bodily sample sample in the presence of a chelating agent or antioxidant. CAP-EPs and PP-EPs extracted from the bodily sample are then hydrolyzed with phospholipase D to form carboxyalkylpyrrole ethanolamine (CAP-ETN) and pentylpyrrole ethanolamine (PP-ETN) derivatives. The amount of carboxyalkylpyrrole ethanolamine (CAP-ETN) and/or pentylpyrrole ethanolamine (PP-ETN) derivatives is then determined by mass spectrometry. The amount of carboxyalkylpyrrole ethanolamine (CAP-ETN) and/or pentylpyrrole ethanolamine (PP-ETN) derivative is determinative of the level of carboxyalkylpyrrole ethanolamine phospholipids (CAP-EPs) and pentylpyrrole ethanolamine phospholipids (PP-EPs) in the sample and indicative of the risk of AMD, tumor progression, or sickle cell disease in the subject.

In some embodiments, the chelating agent or antioxidant can include at least one of ethylenediaminetetraacetic acid (EDTA) or butylated hydroxytoluene (BHT).

In other embodiments, the bodily sample can include at least one of blood, plasma, or sera.

In some embodiments, the method can be used to characterize the risk of a subject developing AMD. The method can include measuring the level of one or more carboxyethylpyrrole ethanolamine phospholipids (CEP-EPs) in a bodily sample of the subject as described herein. The level of the one or more CEP-EPs measured can then be compared to a control value. The subject can be characterized as at greater risk of developing AMD if the level of the one or more CEP-EPs measured is greater that than the control value or the subject can be characterized as at lesser risk of developing AMD if the level of one or more CEP-EPs measured is not greater than the control value.

In other embodiments, the method can be used to characterize the risk of CAP-associated tumor progression in a subject. The method includes measuring the level of one or more CAP-EPs in a bodily sample of the subject as described herein. The level of the one or more CAP-EPs measured can be compared to a control value. The subject can be characterized as at greater risk of developing CAP-associated tumor progression if the level of the one or more CAP-EPs measured is greater that than the control value or the subject can be characterized as at lesser risk of developing CAP-associated tumor progression in a subject if the level of one or more CAP-EPs measured is not greater than the control value.

In other embodiments, the method can be used for identifying a candidate compound for treating or reducing the risk of developing a CAP associated disease or disorder. The method includes measuring the level one or more CAP-EPs in a bodily fluid of a subject at a first time before administering the test compound and at a second time after administering the test compound as described herein. The increase in the level of one or more CAP-EPs measured between the first and second time can be compared to a control value. The test compound can be identified as a candidate compound for treating or reducing the risk of developing CAP associated disease or disorder if the increase in the level of one or more CEP-EPs measured is less than the control value.

Other embodiments relate to a method used characterize the risk of sickle cell disease in a subject. The method includes measuring the level of one or more CAP-EPs and/or PP-EPs in a bodily sample of the subject as described herein. The level of the one or more CAP-EPs and/or PP-EPs measured can be compared to a control value. The subject can be characterized as at greater risk of developing sickle cell disease if the level of the one or more CAP-EPs and/or PP-EPs measured is greater that than the control value or the subject can be characterized as at lesser risk of developing sickle cell disease in a subject if the level of one or more CAP-EPs or PP-EPs measured is not greater than the control value.

### BRIEF DESCRIPTION OF THE DRAWINGS

The foregoing and other features of the present application will become apparent to those skilled in the art to which the present application relates upon reading the following description with reference to the accompanying drawings, in which:

FIGS. 1(A-D) illustrates LC-MS/MS chromatograms showing that PLD (Streptomyces chromofuscus) can effectively hydrolyze CEP-PE and d₄-CEP-PE to CEPETN and d₄-CEP-ETN, respectively. CEP-PE (panels A₀-D₀) or d₄-CEP-PE (panels A₄-D₄) standards were treated either without (panels A₀, B₀, A₄, B₄) or with (panels C₀, D₀, C₄, D₄) PLD for 2 h. Then CEP-ETN and d₄-CEP-ETN were analyzed by MRM transitions m/z 184.3→124.2 or 188.3→128.2, respectively.

FIGS. 2(A-D) illustrate images, plots, and graphs showing the effects of CEP-DPPE on tube formation by HUVECs. (A) Assay used PBS as negative control. HUVEC cells cocultured with Matrigel in the absence (control) or presence of test samples: 60 ng/mL VEGF, 1.3 μM CEP-dipeptide, 0.5 μM CEP-DPPE, and 0.5 μg/mL of CEP in CEP-BSA and CEP-MSA protein derivatives for 8 h. Right: quantification of tube formation assay reported as means±S.D. (n=3); (B) Assay used DPPE as negative control. Left: representative micrographs of cells with various treatments as indicated. Right: quantification of tube formation assay reported as means±S.D. (n=3); (C) Effect of oxPAPC (30.0 μg/ml), a TLR2/TLR4 inhibitor, on the tube-formation ability of HUVEC cells in the absence and in the presence of various concentrations of CEP-DPPE. Results shown are means of duplicate cultures±S.D. and are representative of two similar experiments. *, p<0.001 versus control (ANOVA); **, p<0.02 (t-test) HUVEC CEP-DPPE treatment versus the respective concentration of CEP-DPPE treatment in the presence of oxPAPC inhibitor; (D) Effect of TLR2 knockdown (kdn) on the tube-formation ability of HUVECs in the absence and in the presence of various concentrations of CEP-DPPE. Results shown are means of duplicate cultures±S.D. and are representative of two similar experiments. *, p<0.005 versus control (ANOVA); **, p<0.03 (t-test) HUVEC CEP-DPPE treatment versus the respective concentration of CEP-DPPE treatment in the presence of oxPAPC inhibitor.

FIGS. 3(A-B) illustrate plots showing (A) Calibration curve of CEP-ETN. Data points represent mean±SD of three independent experiments; and (B) LC-MS/MS results indicate that CEP-ETN levels were significantly elevated in human plasma samples from AMD patients (n=10) vs normal plasma samples (n=7). Results are presented as mean±SD.

FIGS. 4(A-G) illustrate LC-MS/MS chromatograms of (A) 20 ng of d₄-CEP-PE internal standard; (B-D) three representative LC-MS/MS chromatograms of CEP-ETN generated by PLD hydrolysis of phospholipids extracted from AMD patients' plasma samples; (E-G) three representative LC-MS/MS chromatograms of healthy control individual's plasma samples.

FIG. 5 illustrates plots showing that CEP-DPPE, but not DPPE affinity binds to recombinant mouse TLR2-Fc fusion protein.

FIGS. 6(A-D) illustrate LC-MS data supports the conclusion that PLD from Streptomyces chromofuscus catalyzes the hydrolysis of CAP-DPPE to deliver the corresponding CAP-ETN. CAP-DPPE standards were treated either with or without PLD overnight. Then CAP-ETN was analyzed by LC-MS in the negative ion model. (A) CEP-DPPE plus PLD. (B) CPP-DPPE plus PLD. (C) CHP-DPPE plus PLD. (D) CHP-DPPE without PLD. Top traces: selected ion chromatograms (SICs) of CEP-ETN (m/z 182.5), CPP-ETN (m/z 196.6) and CHP-ETN (m/z 252.5) detected in the reaction mixture in the negative ion mode. Bottom traces: total ion chromatograms of the reaction mixtures.

FIG. 7 illustrates MRM chromatographs from LC/MS/MS analysis of a mixture of all eight authentic standards (CEP-ETN, CEP-ETN-d4, CPP-ETN, CPP-ETN-d4, CHP-ETN, CHP-ETN-d4, PP-ETN and PP-ETN-d4) in the positive ion mode using 0.1% formic acid as additive.

FIG. 8 Representative LC-MS/MS chromatograms of CAP-ETN and PP-ETN generated by PLD hydrolysis of phospholipids extracted from human blood plasma samples from hospitalized patients with SCD.

FIG. 9 is representative LC-MS/MS chromatograms of CAP-ETN and PP-ETN generated by PLD hydrolysis of phospholipids extracted from human blood plasma samples from clinic patients with SCD.

FIGS. 10(A-D) illustrate levels of pyrroles in human plasma samples from SCD clinic patients (SC-C, n=5) and hospitalized SCD patients (SC-H, n=6) determined by LC-MS/MS: (A) CEP-ETN, **=p<0.002; (B) CPP-ETN, **=p<0.002; (C) CHP-ETN, “NS”-not significant; (D) PP-ETN, *=p<0.05.

### DETAILED DESCRIPTION

It should be understood that the present invention is not limited to particular methods, reagents, compounds, compositions or biological systems, which can, of course, vary. It should also to be understood that the terminology used herein is for the purpose of describing particular aspects of the present invention only, and is not intended to be limiting. Unless defined otherwise, all technical and scientific terms used herein have the same meaning as commonly understood by one of ordinary skill in the art to which the present invention pertains. Although any methods and materials similar or equivalent to those described herein can be used in the practice of the present invention, the preferred materials and methods are described herein.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

All publications, patent applications, patents, and other references mentioned herein are incorporated by reference in their entirety.

Embodiments described herein relate to methods for the detection and measurement of carboxyalkylpyrrole ethanolamine phospholipids (CAP-EPs) and/or pentylpyrrole ethanolamine phospholipids (PP-EPs) or to their use in the clinical assessment of age-related macular degeneration (AMD) risk, CAP-associated tumor progression, sickle cell disease, and CAP associated disease or disorders as well as for monitoring the efficacy of associated therapeutic interventions.

Phospholipids that contain polyunsaturated fatty acids (PUFAs), e.g., linoleic acid (LA), arachidonic acid (AA) or docosahexaenoic acid (DHA), are cellular targets of free radical-induced oxidative damage that initially yields a variety of lipid hydroperoxides. Fragmentation of these hydroperoxides generates oxidatively truncated aldehydes including γ-hydroxy-α,β-unsaturated aldehydes (γ-hydroxyalkenals). γ-Hydroxyalkenals are reactive electrophiles that modify biological nucleophiles, such as proteins, DNA and ethanolamine phospholipids, to form adducts that have been demonstrated to be involved in several pathological processes.

It was found that 4-hydroxy-2-hexenal (4-HHE), derived from DHA, and phospholipids esterified with γ-hydroxy-α,β-unsaturated aldehydes, 9-hydroxy-12-oxododec-10-enoic acid (HODA), 5-hydroxy-8-oxooct-6-enoic acid (HOOA) and 4-hydroxy-7-oxhept-5-enoic acid (HOHA), which are formed upon free radical-induced oxidation of phospholipids containing LA, AA, or DHA, respectively, generate the corresponding ethyl pyrrole, 2-(ω-carboxyheptyl)pyrrole (CHP), 2-(ω-carboxypropyl)pyrrole (CPP), and carboxyethylpyrrole (CEP) derivatives of proteins.

CEP epitopes were found to be more abundant in age-related macular degeneration (AMD) than in normal Bruch's membrane/retinal pigmented epithelium/choroid tissues. For example CEP-EPs derived from HOHA-PLs were found to be present in human blood at 4.6-fold higher levels in AMD plasma than in normal plasma. Moreover, it was found that uniformly elevated blood levels of CEP-EP similar to mean levels in blood from age-related macular degeneration (AMD) patients were detected in plasma from SCD patients hospitalized to treat a sickle cell crisis. Plasma levels of CPP-EPs from SCD clinic patients were four-fold higher than those of SCD patients hospitalized to treat a sickle cell crisis. PP-EP concentration in plasma from SCD clinic patients were nearly 4.8-fold higher than its level in plasma samples from SCD patients hospitalized to treat a sickle cell crisis.

The level of carboxyalkylpyrrole or pentylpyrrole ethanolamine phospholipids (PP-EPs or CAP-EPs), in bodily samples of subjects have previously not been detectable (and/or quantifiable) by mass spectrometry. Multiple reactive aldehydes are generated from peroxidation of different PUFAs in vivo, which requires careful analysis with mass spectrometry. Liquid chromatography-tandem MS (LC/MS/MS) not only allows the use of internal isotopomer standards to correct for different sources of analytical variability but also the use of chromatography to separate different analytes to identify specific modifications of EPs, but at the same time allows the simultaneous measurement of various pyrrole-modified EPs. Isotope-labeled internal standard can be used to minimize variable sensitivity owing to any matrix effects for the biological samples. Because EPs are found in vivo in various forms, mainly 1,2-diacyl, 1-alkyl-2-acyl, and 1-vinyl-2-acyl forms, each of which can potentially differ in acyl chain length and unsaturation, a complex mixture of pyrrole modified EPs with different molecular weights are generated. The amount of each species can be low, possibly below the lowest limit of mass spectrometric detection. To overcome this problem, we previously employed phospholipase A2 to convert the putative complex mixture of isolevuglandin-modified EPs into a much simpler mixture by hydrolyzing the sn-2 acyl chain releasing 2-lysophospholipid. However, this strategy only removes the differences in mass related to the sn-2 position. To provide an even more sensitive index of oxidative stress, phospholipase D (PLD) from Streptomyces chromofuscus can be used to remove all of the differences in mass related to the glycerophospholipid moieties by hydrolyzing various EP pyrrole derivatives to their corresponding modified ethanolamine (ETN) derivatives. This maneuver greatly simplifies the number of total masses that must be quantified releasing a single CAP-ETN or PP-ETN from each mixture of EPs.

Detection and quantification of CAP-ETN or PP-ETN be used determine the levels of CAP-EP and PP-EP in bodily samples (e.g., blood). The detected levels of CAP-EP and PP-EP can be used in the clinical assessment of age-related macular degeneration (AMD) risk, CAP-associated tumor progression, sickle cell disease, and for monitoring the efficacy of associated therapeutic interventions.

In some embodiments, a method of characterizing the risk that a subject will develop AMD (e.g., wet or dry AMD), tumor progression, sickle cell disease or an analogous disease, which involves oxidative damage to tissues from oxidation of DHA containing phospholipids is provided. The method includes the steps of measuring the level of one or more CAP-EPs and/or PP-EPs in a bodily sample or fluid of the subject; comparing the level of the one or more CAP-EPs and/or PP-EPs measured to a control value; and characterizing the subject as at greater risk of developing AMD, sickle disease, or disease which involves oxidative damage to tissues if the level of the one or more CAP-EPs and/or PP-EPs measured is greater that than the control value or characterizing the subject as at lesser risk of developing a disease which involves oxidative damage to tissues from oxidation if the level of one or more CAP-EPs and/or PP-EPs measured is not greater than the control value.

The subject can be any human or other animal to be tested for characterizing its risk of having or developing AMD, tumor progression, sickle cell disease, or an analogous disease which involves oxidative damage to tissues. In certain embodiments the subject is an apparently healthy subject. “Apparently healthy”, as used herein, means individuals who have not previously being diagnosed as having any signs or symptoms indicating the presence of AMD, tumor progression, sickle cell disease or an analogous disease described herein. Apparently healthy individuals also do not otherwise exhibit symptoms of disease. In other words, such individuals, if examined by a medical professional, would be characterized as healthy and free of symptoms of disease. In certain embodiments, the subject is not suffering from diabetes.

### Determining the Levels of CAP-EPs and PP-EPs in Bodily Samples of the Test Subject

The bodily samples or fluids, which may be obtained from the subject and used as test samples in the detection or diagnostic methods include, but are not limited to blood, serum, or plasma. In certain embodiments, the samples are blood or blood fractions, such as serum and plasma. The sample may be a blood sample expressly obtained for the assays described herein or a blood sample obtained for another purpose which can be subsampled for the assays described herein. The sample may be fresh blood or stored blood (e.g., in a blood bank) or blood fractions.

The bodily samples can be obtained from the subject using sampling devices such as swabs, syringes, or other sampling devices used to obtain liquid and/or solid bodily samples either invasively (i.e., directly from the subject) or non-invasively. These samples can then be stored in storage containers. The storage containers used to contain the collected sample can comprise a non-surface reactive material, such as polypropylene. The storage containers should generally not be made from untreated glass or other sample reactive material to prevent the sample from becoming absorbed or adsorbed by surfaces of the glass container.

In one embodiment, the biological sample is whole blood. Whole blood may be obtained from the subject using standard clinical procedures. In another embodiment, the biological sample is plasma. Plasma may be obtained from whole blood samples by centrifugation of anti-coagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

Collected samples may be stored under refrigeration temperature. For longer storage times, it is desirable that the collected sample be frozen to retard decomposition and facilitate storage. For example, samples obtained from the subject can be stored in a falcon tube and cooled to a temperature of about −70° C. Advantageously, the collected bodily sample can be stored in the presence of a chelating agent, such as ethylenediaminetetraacetic acid (EDTA), and/or an antioxidant, such as butylated hydroxytoluene (BHT), to inhibit oxidation of the sample.

Bodily samples obtained from the subject can then extracted with a solvent, such as an organic solvent, to isolate and/or separate phospholipids including CAP-EPs or PP-EPs from the bodily sample. In some embodiments, the phospholipids can be extracted or separated from the bodily sample by contacting the bodily sample with at least one organic solvent under conditions such that an extracted sample is produced. The solvent can include any chemical useful for the removal (i.e., extraction) of a lipid from a bodily sample. For example, where the bodily sample comprises plasma, the solvent can include a chloroform/methanol mixture (e.g., ½ v/v). It will be appreciated by one skilled in the art that the solvent is not strictly limited to this context, as the solvent may be used for the removal of lipids from a liquid mixture, with which the liquid is immiscible in the solvent. Those skilled in the art will further understand and appreciate other appropriate solvents that can be employed to extract lipids from the bodily sample.

The solvent can include solvent mixtures comprising miscible, partially miscible, and/or immiscible solvents. For example, the solvent can comprise a mixture of chloroform and methanol. The solvent can also be combined with other solvents or liquids, which are not useful for the removal of the lipids. The other solvents in the solvent mixture can act as carriers, which facilitate mixing of the solvent with the bodily sample or transfer of the extracted lipids from the bodily sample.

In other embodiments, the bodily sample can be extracted in the presence of a chelating agent, such as ethylenediaminetetraacetic acid (EDTA), and/or an antioxidant, such as butylated hydroxytoluene (BHT), to inhibit oxidation of the sample and extracted lipid.

Following extraction of the phospholipids from the bodily sample, CAP-EPs and PP-EPs in the extract, are hydrolyzed with an enzyme that forms CAP-ETN and PP-ETN derivatives that are capable of being readily detected and quantified by mass spectrometry. T enzyme can include a phospholipase that hydrolyzes phospholipids, such as phosphatidylethanolamine, of the CAP-EPs and PP-EPs to CAP-ETN and PP-ETN derivatives. In some embodiments, the phospholipase can include phospholipase D (PLD) from Streptomyces chromofuscus that selectively cleaves the the phospholipid of CAP-EPs and PP-EPs after the phosphate, releasing phosphatidic acid and an alcohol.

Advantageously, the CAP-EPs and PP-EPs can be hydrolyzed in the presence of a chelating agent, such as ethylenediaminetetraacetic acid (EDTA), and/or an antioxidant, such as butylated hydroxytoluene (BHT), to inhibit oxidation of the CAP-EPs and PP-EPs as well as the resulting CAP-ETN and PP-ETN derivatives.

Following hydrolysis of the CAP-EPs and PP-EPs to form CAP-ETN and PP-ETN derivatives, the CAP-ETN and PP-ETN derivatives can be extracted with a solvent, such as an organic solvent, to further isolate and/or separate the CAP-ETN and PP-ETN derivatives from the enzyme. In some embodiments, the CAP-ETN and PP-ETN derivatives can be extracted with at least one organic solvent. The solvent can include, for example, methanol, chloroform, or a chloroform/ methanol mixture (e.g., ½ v/v). It will be appreciated by one skilled in the art that the solvent is not strictly limited to this context, as the solvent may be used for the removal of CAP-ETN and PP-ETN derivatives from a liquid mixture, with which the liquid is immiscible in the solvent. Those skilled in the art will further understand and appreciate other appropriate solvents that can be employed to extract CAP-ETN and PP-ETN derivatives.

Following solvent extraction, the CAP-ETN and PP-ETN derivatives derivatives can be further purified by liquid chromatography (LC) prior to quantification using mass spectrometry. Liquid chromatography removes impurities and may be used to concentrate the CAP-ETN and PP-ETN derivatives derivatives for detection. Traditional LC relies on chemical interactions between sample components (e.g., CAP-ETN and PP-ETN derivatives) and a stationary phase such as a column packing. Laminar flow of the sample, mixed with a mobile phase, through the column is the basis for separation of the components of interest. The skilled artisan understands that separation in such columns is a partition process.

In various embodiments, one or more of the purification and/or analysis steps can be performed in an automated fashion. By careful selection of valves and connector plumbing, two or more chromatography columns can be connected as needed such that material is passed from one to the next without the need for any manual steps. In certain embodiments, the selection of valves and plumbing is controlled by a computer pre-programmed to perform the necessary steps. The chromatography system can also be connected in-line to the detector system, e.g., an MS system. Thus, an operator may place a tray of hydrolyzed and purified samples in an autosampler, and the remaining operations are performed under computer control, resulting in purification and analysis of all samples selected. In one embodiment, a diverter valve is placed in-line between the LC column and the interface with the MS. The diverter valve directs the LC effluent into a waste container until slightly prior to the time expected peak retention). This prevents the solvent front and other impurities from being passed into the MS device.

As used here, “in-line” refers to a configuration in which the LC and the ionization/injection device for the first MS quadropole are functionally connected in order that the LC effluent passes directly into the first MS device. “In-line” configurations may include a selector valve such that the effluent from two or more LC columns may be directed individually into the MS device and, optionally, to a waste container. Such a configuration is useful for a high throughput system and reduces the analysis time required for a large number of samples. High throughput systems may be designed in which an autosampler initiates LC purifications on the two or more LC columns at staggered intervals. In this way, the purified CAP-ETN and PP-ETN derivatives peak is eluted from each LC column at a known interval. In certain embodiments, the purified CAP-ETN and PP-ETN derivatives peaks eluting from the two or more LC columns are directed into the MS device in rapid succession, but with sufficient temporal separation that individual measurements are not compromised. Such a high throughput system reduces the amount of “idle-time” for MS detection attributable to the LC procedure, which typically requires more time than the MS analysis.

By contrast, “off-line” refers to a configuration that requires manual intervention to transfer the LC effluent to the MS device. Typically, the LC effluent is captured by a fractionator and must be manually loaded into a MS device or into an autosampler for subsequent MS detection. Off-line configurations are useful, but less desirable because of the increased time required to process large numbers of samples.

CAP-ETN and PP-ETN derivatives purified by LC are conveniently detected and quantified by mass spectrometry (MS). The CAP-ETN and PP-ETN derivatives-containing effluent from the LC is injected into an ionization chamber of the MS in which a first (parent) ion is produced. The parent ion may be detected directly in a first MS, or it may be isolated by the first MS, fragmented into characteristic daughter ions, and one or more of the daughter ions detected in a second MS (i.e., tandem MS).

The term “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”).

As used herein, the term “ionization” refers to the process of generating an analyte ion having a net electrical charge equal to one or more electron units. Negative ions are those having a net negative charge of one or more electron units, while positive ions are those having a net positive charge of one or more electron units.

The ions may be detected using several detection modes. For example, selected ions may be detected using a selective ion monitoring mode (SIM) which includes multiple reaction monitoring (MRM) or selected reaction monitoring (SRM). Alternatively, ions may be detected using a scanning mode.

In some embodiments, the mass-to-charge ratio is determined using a quadrupole analyzer. For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as both a “mass filter” and as a “mass detector” for the ions injected into the instrument.

“Tandem mass spectrometry,” or “MS/MS” is employed to enhance the resolution of the MS technique. In tandem mass spectrometry, a parent ion generated from a molecule of interest may be filtered in an MS instrument, and the parent ion subsequently fragmented to yield one or more daughter ions that are then analyzed (detected and/or quantified) in a second MS procedure.

Collision-induced dissociation (“CID”) is often used to generate the daughter ions for further detection. In CID, parent ions gain energy through collisions with an inert gas, such as argon, and subsequently fragmented by a process referred to as “unimolecular decomposition.” Sufficient energy must be deposited in the parent ion so that certain bonds within the ion can be broken due to increased vibrational energy.

By careful selection of parent ions using the first MS procedure, only ions produced by certain analytes of interest are passed to the fragmentation chamber to generate the daughter ions. Because both the parent and daughter ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

The mass spectrometer typically provides the user with an ion scan; that is, the relative abundance of each m/z over a given range (e.g., 10 to 1200 amu). The results of an analyte assay, that is, a mass spectrum, can be related to the amount of the analyte in the original sample by numerous methods known in the art. For example, given that sampling and analysis parameters are carefully controlled, the relative abundance of a given ion can be compared to a table that converts that relative abundance to an absolute amount of the original molecule. Alternatively, molecular standards (e.g., internal standards and external standards) can be run with the samples, and a standard curve constructed based on ions generated from those standards. Using such a standard curve, the relative abundance of a given ion can be converted into an absolute amount of the original molecule. In certain preferred embodiments, an internal standard is used to generate a standard curve for calculating the quantity of isolevuglandin and/or levuglandin phospholipid derivatives. Numerous other methods for relating the presence or amount of an ion to the presence or amount of the original molecule are well known to those of ordinary skill in the art.

The skilled artisan will understand that the choice of ionization method can be determined based on the analyte to be measured, type of sample, the type of detector, the choice of positive versus negative mode, etc. Ions can be produced using a variety of methods including, but not limited to, electron ionization, chemical ionization, fast atom bombardment, field desorption, and matrix-assisted laser desorption ionization (MALDI), surface enhanced laser desorption ionization (SELDI), photon ionization, electrospray ionization, and inductively coupled plasma. Electrospray ionization is a preferred ionization method. The term “electrospray ionization,” or “ESI,” as used herein refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated to prevent condensation and to evaporate solvent. As the droplets get smaller the electrical surface charge density increases until such time that, the natural repulsion between like charges causes ions as well as neutral molecules to be released.

Desirably, the effluent of the LC is injected directly and automatically (i.e., “in-line”) into the electrospray device. In certain embodiments, the CAP-ETN and PP-ETN derivatives contained in the LC effluent is first ionized by electrospray into a parent ion of about 50 to about 900 m/z. The first quadropole of the MS/MS is tuned to be a mass filter for the CAP-ETN and PP-ETN derivative parent ions (and/or the internal standard).

Parent ion(s) passing the first quadropole are then ionized and/or fragmented prior to passing into the second quadropole. In certain embodiments, the ions are collided with an inert gas molecule in a process of collision-induced dissociation (CID). Examples of inert gases include, for example, argon, helium, nitrogen, etc. Desirably, the CAP-ETN and PP-ETN derivative parent and daughter ions can be subsequently detected.

It is desirable to use one or more standards for calibration and quantification purposes. Internal and external standards are commonly used for these purposes. Internal standards are typically analogs of the compound(s) of interest that are expected to react similarly during all extraction and quantification steps. A known amount of an internal standard is typically added to each sample early in the processing in order to account for any loss of compound during any processing step. External standards typically consist of samples containing a known quantity of the compound of interest, or an analog, which are processed in parallel with the experimental samples. External standards are often used to control for the efficiency of the various processing steps. Finally, calibration standards are used to quantify the amount of the compound of interest in each experimental and external control sample. Typically, a series of calibration standards containing varying known amounts of the compound(s) of interest are injected directly into the detection device (i.e., the MS). Calibration standards are used to generate a standard curve, against which the experimental samples are quantified. These standards also may be used to determine limits of detection for any particular detection methodology.

In certain embodiments, a mass spectrometry-based assay to determine the presence and/or level of one or more CAP-EP and PP-EPs can include the use of a stable labeled isotope internal standard. The stable labeled isotopes available to incorporate in a given molecule (drug or drug metabolite) are deuterium (2H or D), 13C and 15N. Generally, because of the abundance of hydrogen in organic molecules, the use of deuterium is preferred compared to 13C and 15N, which are generally more expensive solutions for stable labelled internal standards.

In certain embodiments, a internal standard will have the same extraction recovery, ionization response in ESI mass spectrometry and the same chromatographic retention time. In some embodiments, a deuterated internal standard can co-elute with the compound to be quantified and contain enough mass increase to show a signal outside the natural mass distribution of the analyte. In certain embodiments, a deuterated internal standard for use in a method of the present invention can include a CAP-ethanol-1,1,2,2-d₄-amine (CAP-ETN-d₄) or PP-ETN-d₄ standard.

In one exemplary embodiment of an LC-MS/MS assay for use in the methods described herein, lipid extracts from 200 μL of a subject's human plasma samples are hydrolyzed by 280 units of PLD (Streptomyces chromofuscus). CAP-ETN-d₄ (20 ng, 0.023 nmol) is added to the sample as internal standard before PLD hydrolysis. Then the CAP-ETN levels in the hydrolysis reaction product mixtures are determined by LC-MS/MS. To minimize possible interference from variations in phospholipolysis efficiency and interference from the complex biological matrices that may vary among samples, a phospholipid internal standard was implemented for the LC-MS/MS analysis. A CAP-ETN-d₄ standard was prepared from L-a-phosphatidylcholine (egg PC) by an enzyme mediated transphosphatidylation. A correction for this trace impurity can be applied during data analysis (vide infra). A calibration curve for CAP-ETN quantification is established with samples containing pure CAP-ETN in HBSS buffer in the concentration range from 2-30 nM. A fixed amount of internal standard, CAP-ETN-d₄ (0.023 nmol), was added to 200 μL of each calibrator standard. The peak area ratio of CAP-ETN to CAP-ETN-d₄ from the LC-MS/MS analysis is ploted against calibrator concentrations to determine CAP-EP and/or PP-EP level or concentration in the sample.

### Comparing the Level of the One or More CAP-EPs or PP-EPs Measured to a Control Value

Levels of the one or more the CAP-EPs or PP-EPs in the bodily sample of the test subject may then be compared to a control value that is derived from levels of the one or more the CAP-EPs or PP-EPs in comparable bodily samples e.g., from the general population or a select population of human subjects. In some embodiments, the diagnosis is made by comparing the amount, concentration or content of the CAP-EPs and/or PP-EPs in a sample obtained from the test subject to the amount of CAP-EPs and/or PP-EPs in samples obtained from subjects lacking the disease, i.e., healthy or normal subjects. Alternatively, the amount, concentration or content of CAP-EPs and/or PP-EPs in the sample may be compared to the amount, concentration or content in corresponding samples which were taken from the test subject for the purpose of determining baseline concentrations of the CAP-EPs and/or PP-EPs, such as for example during an early screening procedure. Accordingly, the control values selected may take into account the category into which the test subject falls. Appropriate categories can be selected with no more than routine experimentation by those of ordinary skill in the art.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having AMD, sickle cell disease, or another disease associated with oxidation of lipids can be based upon which group his or her test value falls.

Control values of one or more the CAP-EPs and/or PP-EPs in biological samples obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each risk predictor that is assayed.

Characterizing the Subject'S Risk of Having or Developing AMD, Tumor Progression, Sickle Cell Disease or an Analogous Disease which Involves Oxidative Damage to Tissues from Oxidation of DHA Containing Phospholipids

The levels of one or more the CAP-EPs or PP-EPs, in the test subject's bodily fluid may be compared to a single predetermined value or to a range of predetermined values. Test subjects whose levels of the one or more CAP-EPs or PP-EPs are above the control value or in the higher range of control values are characterized as having a greater risk of having or developing AMD, sickle cell disease or another disease associated with oxidation of DHA-containing lipids, than test subjects whose levels of the one more the CAP-EPs or PP-EPs are at or below the control value or in the lower range of control values. Moreover, the extent of the difference between the subject's the CAP-EP and/or PP-EP levels and the control value is also useful for characterizing the extent of the risk and thereby, determining which subjects would most greatly benefit from certain therapies. In certain embodiments, the characterizing step includes characterizing the subject as having a less than a 60% chance of developing a disease associated with oxidation of DHA-containing lipids. In other embodiments, the characterizing step includes characterizing the subject as having a greater than a 60% chance of developing a disease associated with oxidation of DHA-containing lipids.

In some embodiments, the present methods are useful for determining if and when therapeutic agents which are targeted at preventing AMD, sickle cell disease, or another disease associated with oxidation of DHA-containing lipids should and should not be prescribed for a patient.

In certain embodiments, a subject's risk profile for AMD or sick cell disease is determined by combining a first risk value, which is obtained by comparing levels of one or more CAP-EPs or PP-EPs in a bodily sample of the subject with levels of said one or more, respective, CAP-EPs or PP-EPs in a control population, with one or more additional risk values to provide a final risk value. Such additional risk values may be determined the subject's age (especially after age of 50), family history of macular degeneration, race, smoking, obesity, diet, cardiovascular disease, and/or cholesterol in a bodily sample from the subject.

In another embodiment, the present methods are used to assess the test subject's risk of developing AMD or sickle cell disease in the future. In one embodiment, the test subject is an apparently healthy individual. In another embodiment, the subject is not otherwise at elevated risk of having AMD or sickle cell disease. In another embodiment, the present methods are used to determine if a subject presenting with vision changes is at risk of developing AMD or sickle cell disease, near term. As used herein, the term “near term” means within one year.

For example, subjects who are at near term risk may be at risk of developing AMD within the following day, 3 months, or 6 months after presenting with vision changes. Such vision changes indicating that a subject may be at risk of developing AMD can include: Vision changes can include the need for brighter light when reading or doing close work; Increasing difficulty adapting to low light levels, such as when entering a dimly lit restaurant; Increasing blurriness of printed words; A decrease in the intensity or brightness of colors; Difficulty recognizing faces; A gradual increase in the haziness of your central or overall vision; Crooked central vision; A blurred or blind spot in the center of your field of vision; Hallucinations of geometric shapes or people, in case of advanced macular degeneration.

The present invention also provides a method for monitoring over time the status of AMD, sickle cell disease, or a disease associated with oxidation of DHA-containing lipids in a subject who has been diagnosed as having the disease. In this context, the method is also useful for monitoring the risk for disease progression or regression in a subject with AMD or sickle cell disease. In one embodiment, the method comprises determining the levels of one or more of the CAP-EPs or PP-EPs in a bodily sample taken from the subject at an initial time and in a corresponding biological sample taken from the subject at a subsequent time. An increase in levels of the one or more CAP-EPs or PP-EPs in a bodily sample taken at the subsequent time as compared to the initial time indicates that the subject's AMD or sickle cell disease has progressed or worsened. A decrease in levels of the one or more CAP-EPs or PP-EPs indicates that the AMD or sickle cell disease has improved or regressed. For those subjects who have already been diagnosed with AMD or sickle cell disease, such method can also be used to assess the subject's risk of having an advanced form of AMD or sickle cell disease. An increase over time in levels of the one or more CAP-EPs or PP-EPs in the subject indicates that a subject's risk of developing an advanced form of AMD or sickle cell disease and related side effects has increased. A decrease over time in levels of the one or more CAP-EPs or PP-EPs in the subject indicates that that the subject's risk of developing an advanced form of AMD or sickle cell disease has decreased.

In another embodiment, the present invention provides a method for evaluating therapy in a subject suspected of having or diagnosed as having AMD, sickle cell disease, or another disease associated with oxidation of DHA-containing lipids. The method comprises determining levels of one or CAP-EPs or PP-EPs in a bodily sample taken from the subject prior to therapy and determining levels of the one or more of the CAP-EPs or PP-EPs in a corresponding bodily sample taken from the subject during or following therapy. A decrease in levels of the CAP-EPs or PP-EPs in the sample taken after or during therapy as compared to levels of the one or more CAP-EPs or PP-EPs in the sample taken before therapy is indicative of a positive effect of the therapy on AMD or sickle cell disease in the treated subject.

Other embodiments described herein relate to characterizing the risk of CAP-associated tumor progression in a subject. The method includes measuring the level of one or more CAP-EPs in a bodily sample of the subject using the LC-MS/MS assay described herein. The method further includes comparing the level of the one or more CAP-EPs measured to a control value; and characterizing the subject as at greater risk of developing CAP-associated tumor progression if the level of the one or more CAP-EPs measured is greater that than the control value or characterizing the subject as at lesser risk of developing CAP-associated tumor progression if the level of one or more CAP-EPs measured is not greater than the control value.

Elevated blood CAP-EP levels are shown to be correlated with tumor progression after failure of radiation therapy or anti-VEGF therapy. Without being bound by theory, it is believed that elevated levels of CAP-EPs may contribute to “resistance” from anti angiogenic therapy, anti-VEGF therapy or radiation therapy. Therefore, in some embodiments, a method of characterizing the risk of CAP-associated tumor progression in a subject provided herein can be applied to subjects who have or are currently undergoing anti angiogenic therapy, anti-VEGF therapy or radiation therapy

### Kits

Another aspect of the invention relates to diagnostic kits and reagents which may be employed in assays to detect the presence of a CAP-EPs or PP-EPs in bodily samples of test subjects for the purpose of characterizing the risk that a subject has or will develop diseases which involve oxidative damage to tissues from oxidation lipids. The diagnostic kit comprises an internal standard. Such internal standard may be employed to generate a standard curve for quantification, preferably a deuterated CAP-ETN-d₄ internal standard, which is used in an LC-MS/MS assay to detect the presence or quantify the amount of CAP-EPs present in a sample. In another embodiment, the kit comprises information useful for determining a subject's risk of cardiovascular disease or a complication. Examples of such information include, but are not limited cut-off values, sensitivities at particular cut-off values, as well as other printed material for characterizing risk based upon the outcome of the assay.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

In this Example, we applied liquid chromatography tandem mass spectrometry (LC-MS/MS) to measure CEP-EP levels in subjects with AMD.

### Materials

1,2-Dipalmitoyl-sn-glycero-3-phosphoethanolamine (DPPE) and egg PC (L-α phosphatidylcholine) were purchased from Avanti Polar Lipids (Alabaster, Ala.). Recombinant mouse TLR2-Fc protein was purchased from R&D systems (Minneapolis, Minn.). Phospholipase D (PLD) from Streptomyces sp. and Streptomyces chromofuscus were obtained from Enzo Life Sciences (Farmingdale, N.Y.). Horseradish peroxidaselabeled goat anti-human IgG Fc antibody was purchased from Millipore (Billerica, Mass.). ABTS solution substrate for horseradish peroxidase was from Invitrogen (Grand Island, N.Y.). 0xPAPC was from InvivoGen (San Diego, Calif.). The fluorenemethyl ester of 3,6-dioxohexanoic acid (DOHA-Fm), CEP modified human serum albumin (CEP-HSA), chicken egg ovalbumin (CEP-CEO) and polyclonal rabbit anti-CEP-KLH antibody, were prepared using well known methods. Mouse anti-human TLR2 LEAF antibody (CD282) was from BioLegend (San Diego, Calif.). Calcein AM and Accutase were from BD Biosciences (San Jose, Calif.). Goat antimouse IgG-AlexaFluor 488 antibody was from Invitrogen (Carlsbad, Calif.). All other chemicals were from Sigma-Aldrich and were analytical grade.

### Synthesis of CEP-DPPE

The CEP derivative of 1,2-dipalmitoyl-sn-glycero-3-phosphoethanolamine (CEP-DPPE) was prepared. In brief, to a solution of DPPE (45 mg, 0.065 mmol) in 500 μL CHCl₃ was subsequently added triethylamine (25 μL, 0.247 mmol) and a solution of DOHA-Fm (22 mg, 0.065 mmol) in 500 μL CHCl₃. The resulting mixture was stirred overnight at room temperature under argon. After the reaction was complete, 1,8-diazabicyclo[5.4.0]undec-7-ene (DBU) (20 μL, 0.13 mmol) was added to the mixture that was stirred for another 3 h. The resulting solution was washed with 2 mL of pH 5.5 sodium phosphate buffer. The organic phase was washed with brine, and dried over magnesium sulfate. Solvent was removed by rotary evaporation under reduced pressure, and the residue was purified by silica gel flash chromatography (CHCl₃: MeOH=10:1, R_{f}=0.25) to give pure CEP-DPPE (37.6 mg, 0.046 mmol, 71%). ¹H NMR for CEPDPPE (CD₃OD: CDCl₃=1:1), 6 7.00 (m, 1H), 6.39 (dd, 1H), 6.24 (m, 1H), 5.55 (m, 1H), 4.87 (m, 4H), 4.73 (dd, J=12.1, 3.3 Hz, 1H), 4.50 (m, 4H), 4.18 (s, 2H), 3.27 (t, J=7.2 Hz, 2H), 2.99 (t, J=7.2 Hz, 2H), 2.68 (t, J=7.5 Hz, 4H), 1.98 (m, 4H), 1.78-1.53 (48H), 1.26 (t, J=6.9 Hz, 6H). ESI-MS: m/z calcd for C₄₄H₇₉NO₁₀P [M−H]−, 812.54; found 812.53.

### Synthesis of CEP-ethanolamines (CEP-ETN and d₄-CEP-ETN)

To a solution of DOHA-Fm (33.6 mg, 0.1 mmol) in 2 mL of CH₂Cl₂, was slowly added ethanolamine or ethanol-1,1,2,2-d4-amine (99 atom % D, C/D/N Isotopes Inc., Quebec, Canada) (0.12 mmol) in 1 mL of methanol. The mixture was stirred at room temperature for 5 h under argon. Then 20 μL of DBU were added, and the mixture was stirred for another 6 h. After removal of solvent, 5 mL of ddH2O were added. The pH of the mixture was adjusted to 3 by addition of 0.1 N HCl, then the solution was extracted three times with 5 mL of chloroform. The combined organic extracts were dried over anhydrous sodium sulfate for 3 h, and evaporated to dryness under reduced pressure. The residue was purified by flash chromatography on a silica gel column (CHCl₃: MeOH=20:1, TLC R_{f}=0.18) to give pure CEP-ETN (11.3 mg, 62%) or d₄-CEP-ETN (12.5 mg, 67%). CEP-ETN: 1H NMR (400 MHz, CD₃OD) 6 6.61 (dd, J=2.7, 1.8 Hz, 1H), 5.99-5.89 (m, 1H), 5.83-5.77 (m, 1H), 3.94 (t, J=5.9 Hz, 2H), 3.72 (t, J=5.9 Hz, 2H), 2.90-2.80 (m, 2H), 2.60 (dd, J=8.4, 6.9 Hz, 2H). ¹³C NMR (100 MHz, CD₃OD) δ 175.73, 131.36, 120.45, 106.58, 104.99, 62.01, 48.34, 33.32, 21.34. D₄-CEP-ETN: 1H NMR (400 MHz, CD₃OD) δ 6.61 (dd, J=2.8, 1.8 Hz, 1H), 5.98-5.89 (m, 1H), 5.80 (ddt, J=3.5, 1.7, 0.8 Hz, ¹H), 2.85 (t, J=7.6 Hz, ²H), 2.67-2.54 (m, ²H). ¹³C NMR (100 MHz, CD₃OD) δ 175.65, 131.33, 120.38, 106.58, 104.98, 33.27, 21.31.

### LC-MS/MS Assay for CEP-PE in Human Plasma—Synthesis of Internal Standard d4-CEP-PE

The internal standard, d₄-CEP-PE was synthesized through a PLD (Streptomyces sp) catalyzed transphosphatidylation reaction. L-α-phosphatidylcholine (egg PC) (9.1 mg, 0.012 mmol) was mixed with 300 μL of ethyl acetate. Then d₄-CEP-ETN (5.8 mg, 0.031 mmol) in 200 μL of 0.2 M sodium acetate buffer (pH=5.6) containing 80 mM CaCl₂ and 80 units of PLD (Streptomyces sp.) were added. The biphasic reaction was vigorously stirred at 37° C. for 3 h and then terminated by extraction with three portions of 500 μL of ethyl acetate. The organic layers were combined, and solvent was evaporated under reduced pressure. The residue was purified by flash silica gel chromatography. Unreacted d4-CEPethanolamine was eluted first with CHCl₃/MeOH (20:1, v/v).

Then pure d₄-CEP-PE (9.3 mg, 72.1%) was eluted with CHCl₃/MeOH (10:1, v/v. R_{f}=0.2). ESI-MS analysis confirmed the generation of d4-CEP-PE. The average molecular weight (M.W.) of d₄-CEP-PE was calculated to be 853.1 according to the average M.W. of egg PC (770.1) plus a mass increment of 83 for CEP modification.

### Human Plasma Sample Preparation

Human blood plasma samples from donors with AMD and age-matched donors with no disease (controls) were provided by the Cole Eye Institute of the Cleveland Clinic. Total lipids were isolated from human blood plasma (from blood collected in the presence of EDTA) as follows: 100 μL of plasma was mixed with 200 μL of acetone in the presence of 0.5 mM butylated hydroxytoluene (BHT) to precipitate proteins. After centrifugation for 20 min at 10,000 rpm, the supernatant was transferred to a clean vial, and was dried under a stream of nitrogen gas. The samples were stored under nitrogen at −80° C. until analysis.

### Lipid Extraction and PLD Hydrolysis

The above samples were extracted by a modified Bligh & Dyer method to isolate phospholipids from non-lipid matrix. In brief, the residue was resuspended in 200 μL of PBS, followed by addition of 750 μL of chloroform/methanol (1:2, v/v) containing 1 mM BHT and 20 ng of internal standard (d₄-CEP PE, 0.023 nmol), followed by addition of 250 μL chloroform and 250 μL aqueous sodium chloride solution (1.5%). The resulting mixture was vortexed vigorously and then centrifuged for 10 min at 3,000 rpm. The lower organic layer was collected, dried under a stream of nitrogen. The residue was stored under argon at −80° C. until analysis.

The isolated lipids were hydrolyzed with PLD (Streptomyces chromofuscus) by a modification of the method described previously for hydrolysis of isolevuglandin modified PEs. Lipids were re-suspended in 50 μL of methanol. Then 450 μL of HBSS buffer (Thermo Scientific, Waltham, Mass.) supplemented with 5 mM CaCl₂ and 0.1 mM EDTA was added. The mixture was incubated at 37° C. for 30 min, and then was sonicated for 10 min. The cloudy lipid mixture was passed through a 0.1 μm polycarbonate filter (20 times) for extrusion using an Avanti Mini- Extruder Set (Avanti Polar Lipids, Inc., Alabaster, Ala.) to generate a homogeneous unilammelar lipid vesicle solution. Then 280 units of PLD (Streptomyces chromofuscus) were added to each sample. The mixture was incubated under argon with gentle shaking at 37° C. overnight. The next day the samples were evaporated to dryness under a stream of nitrogen for 2 h. The residue was then reconstituted with 100 μL of methanol under argon, and stored at −80° C. before LC-MS/MS analysis. Twenty μL of above solutions were injected into the LC-MS/MS for each analysis.

### LC-MS/MS Analysis

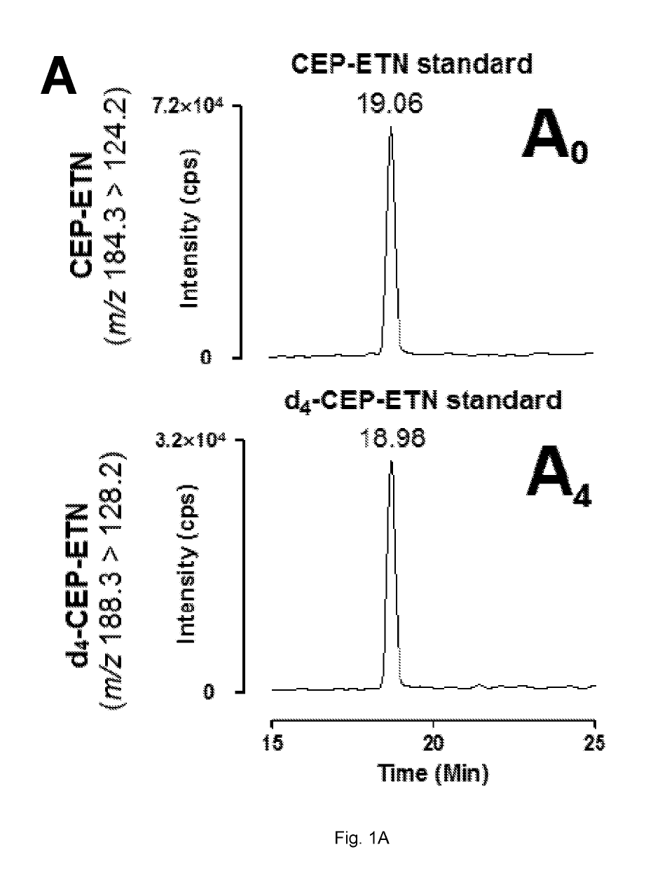

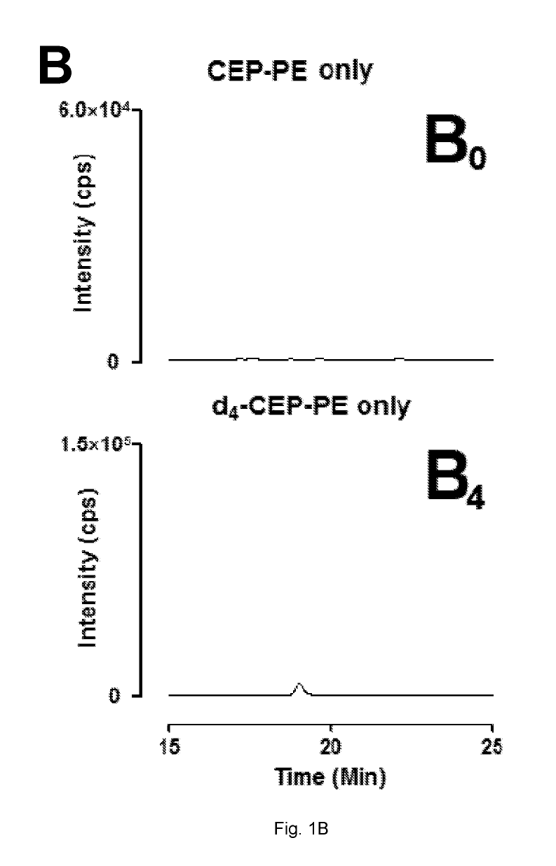

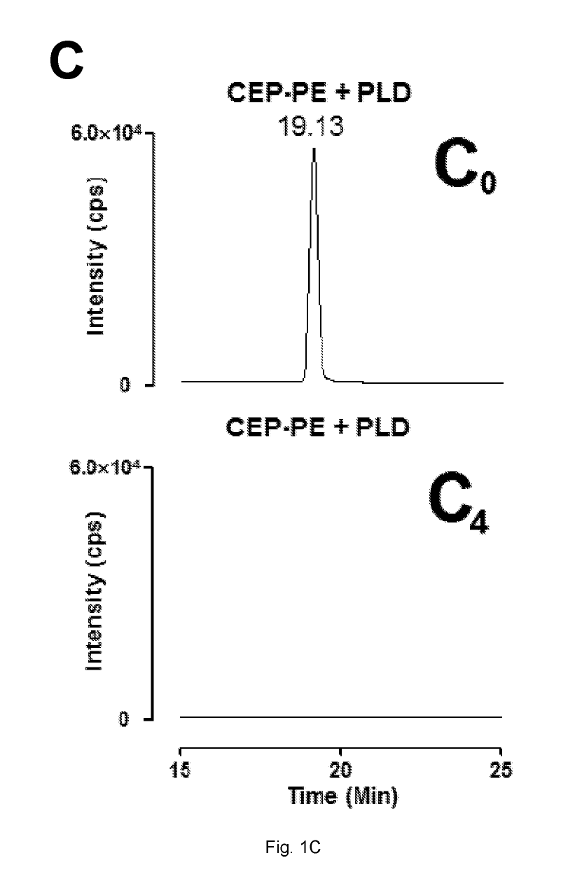

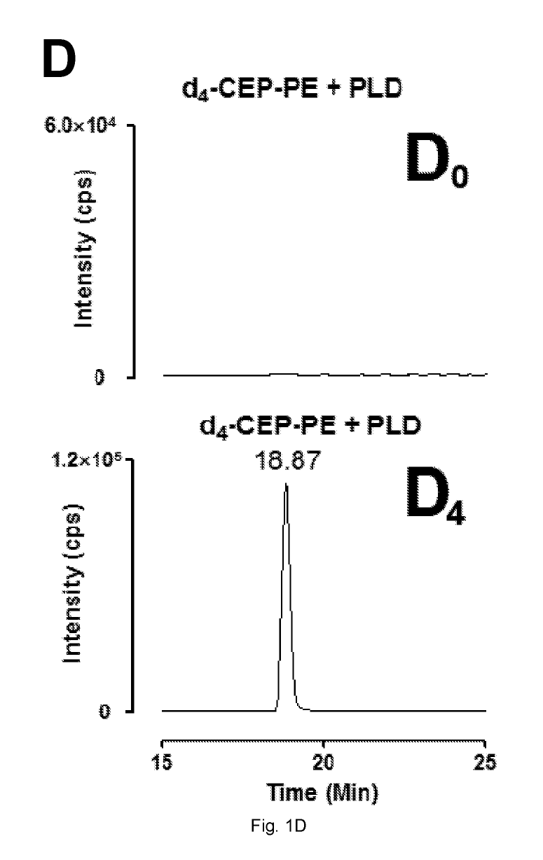

Mass spectrometric analyses of CEP-ETN and d₄-CEP-ETN were performed on a Quattro Ultima triplequadrupole mass spectrometer (Micromass, Wythenshawe, UK) equipped with a Waters Alliance 2690 HPLC system (pump and autosampler) (Waters, Milford, Mass.). Chromatographic separation was achieved using a 150×2.0 mm i.d. Prodigy ODS-5 μm column (Phenomenex). Mobile phase A was 0.1% formic acid in water and mobile phase B was 0.1% formic acid in methanol. The HPLC gradient steps were set as follows: 0-5 min, isocratic at 5% solvent B; 5-22 min, linear gradient from 5 to 100% B; 22-30 min, isocratic at 100% B; 30-31 min, linear gradient from 100 to 5% B; 31-40 min, isocratic at 5% B. The flow rate employed was 200 μL/min. The analytes were measured mass spectrometrically in the positive ion mode with the source temperature at 120° C. the desolvation temperature at 250° C. the drying gas N2 at 450 L/h, a cone gas flow rate of 70 L/h, and multiplier at 600, collision gas at 5 psi and collision energy at 30 eV. Multiple reaction monitoring (MRM) of the transitions 184.3→124.2 and 188.3→128.2 was used to analyze CEP-ETN and d₄-CEP-ETN, respectively (FIGS. 1A and 1A₄). No interference from the isobaric phosphocholine (exact mass: 183.07) was detected.

### HUVEC Tube Formation Assays

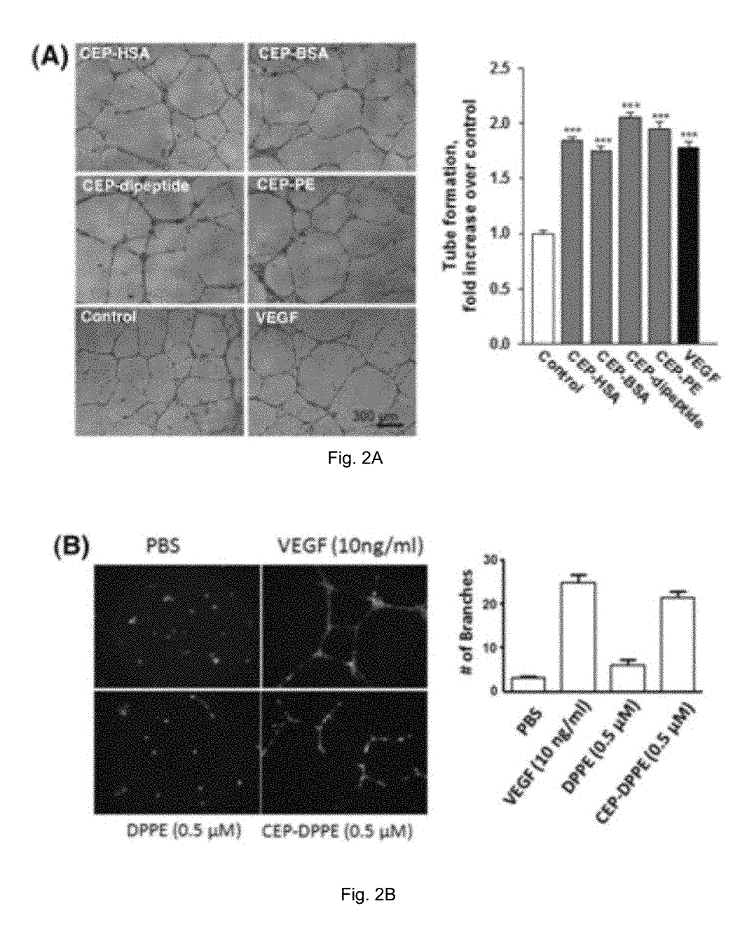

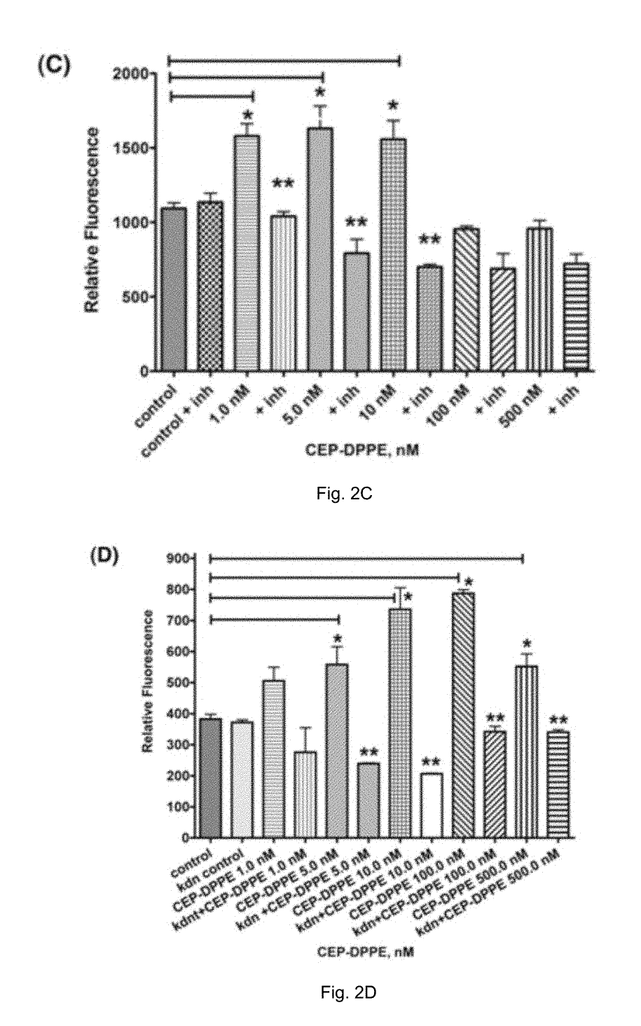

For comparing the ability of various CEP-derivatives to promote tube formation (FIG. 2A) HUVECs were seeded on Matrigel-coated 48 well plates (BD Bioscience). The DMEM-F12 medium (Media lab, Cleveland Clinic, Cleveland, Ohio) was supplemented with various CEP-derivatives as indicated and incubated with cultured HUVECs at 37° C. for 8 h. Tube formation by HUVECs was observed using a phase contrast inverted microscope. Two independent assays were performed by two operators, one used PBS as control and the other used DPPE vesicles as control. The data were quantified either by measuring the length of tubes or by the number of branches. For testing the effects of oxPAPC, a TLR2 inhibitor, (FIG. 2C) or TLR2 knockdown (FIG. 2D) on the ability of CEP-PE to promote tube formation, growth factor-reduced (GFR) basement membrane extract (Cultrex BME, Trevigen, Gaithersburg, Md.; 175 μl/well) was added to the wells of an ice-cold 48-well plate and then incubated at 37° C. for 1 h to allow the gel to solidify. Overnight-starved, trypsin detached and extensively washed HUVECs (2.5×104 cells/well; Promocell, GmbH, Heidelberg, Germany) in a basal Growth Medium 2 (GM2; Promocell, GmbH) were seeded onto the BME coated wells. For testing the effect of a TLR2 inhibitor (FIG. 2C), HUVECs, untreated or treated with 30.0 μg/ml of oxPAPC (InvivoGen; San Diego, California) for 1 h prior to the experiment were challenged with a basal medium alone (GM2) or a basal medium (GM2) containing 1-500 nM CEP-DPPE for at least 16-18 h in 5% CO₂/95% air at 37° C. The following day, the cells were stained with Calcein AM solution (BD Biosciences; San Jose, Calif., 1:100 dilution of a stock 1 mg/ml solution in DMSO in PBS) by adding 50 μl of working solution to each well. After incubating HUVECs for 1 h in 5% CO₂/95% air at 37° C., images of the HUVECs (untreated, treated with CEP-DPPE and treated with CEP-DPPE and 30.0 μg/ml of oxPAPC) were obtained using a Leica DMI 6000 B inverted microscope (5× magnification, FITC filter, Leica Microsystems Wetzlar, Germany) equipped with a Retiga EXI camera (Q-imaging, Vancouver, British Columbia). Image analysis was performed using Metamorph Imaging Software (Molecular Devices, Downington, PA) in an “Angiogenesis Tube Formation” mode. For testing the effect of TLR2 knockdown (FIG. 2D), HUVECs and HUVECs with a transient TLR2 gene knockdown (24 h, see below) were starved for 12 h in GM2 basal medium, treated with Accutase (BD Biosciences; San Jose, Calif.) as suggested by the manufacturer and harvested from the plates using a rubber policeman. They were further thoroughly washed three times with GM2 basal medium, counted and seeded onto BME coated wells. HUVECs and HUVECs with transient TLR2 knockdown were challenged with a basal medium (GM2) alone or GM2 cell culture media containing 1-500 nM CEP-DPPE for 12 h in 5% CO₂/95% air at 37° C. The cells were then stained with Calcein AM solution and images were obtained and processed as described above.

### Transient TLR2 Knockdown

HUVECs were seeded in six-well plates at a density of 0.27×10⁶ cells/well in antibiotic-free normal GM2 growth medium to achieve 60% to 70% confluence on day 2. For transfection of each well, 6-12 μL of TLR2 siRNA duplex (Qiagen; 0.18-0.36 μg) was diluted in 100 μL HBSS and was labeled as A. Then 6-12 μL transfection reagent (HiPerFect; Qiagen; Valencia, Calif.) was diluted in 100 μl of HBSS and labeled as B. Solutions A and B were mixed together and incubated at room temperature for 45 min. The cell monolayer was rinsed in basal GM2 medium, and the siRNA-transfection mix was added dropwise onto the monolayer in GM2 containing 2% FBS and incubated for 3 h in 5% CO₂/95% air at 37° C. Fresh GM2 medium containing 10% FBS was added on the monolayer without removing the transfection mixture and was incubated for an additional 12 h in 5% CO₂/95% air at 37° C.

### Flow Cytometry

Monolayers of normal HUVECs and HUVECs with a transient TLR2 gene knockdown were detached from the plates by Accutase cell detachment solution (BD Biosciences; San Jose, Calif.). The cells were then washed with HBSS containing 1% FCS (PBS FCS) and then were incubated with 2.0 μg of unconjugated primary mouse anti-human TLR2 LEAF antibody (CD282, BioLegend, San Diego, Calif.) in 0.2 ml PBS-FCS for 45 min at 4° C. After washing three times with PBS-FCS, the cells were incubated with the secondary goat antimouse IgG-AlexaFluor 488 (Invitrogen, Carlsbad, Calif.) for 45 min at 4° C. Normal and transiently transfected HUVECs were then washed with PBS-FCS and then were analyzed on a BD LSR II flow cytometer (BD Biosciences; San Jose, Calif.). The FACS data on HUVEC with transient TLR2 gene knockdown showed that TLR2 was completely absent in 20-30% of the cells.

### Competitive Binding of CEP-PE vs CEP-HSA to Recombinant Mouse TLR2-Fc Protein

Small unilamellar vesicles of CEP-DPPE (125 μM) in phosphate buffered saline (PBS) were prepared by extruding (20 times) the hydrated phospholipids through a 0.1 μm polycarbonate filter using an Avanti Mini-Extruder (Avanti Polar Lipids, Inc., Alabaster, Ala.). The resulting solution was diluted to various concentrations by addition of PBS. To a 96-well ELISA plate, 100 μl of 5 μM CEP in CEP-HSA protein in PBS solution was added into each well and the plate was incubated at 37° C. for 1 h. PBS (100 μl) was used as control. Then the wells were washed with PBS and the non-binding sites on wells were blocked with 350 μL of 4% bovine serum albumin (BSA) in PBS buffer for 1 h at 37° C. blocking was completed, a mixture of recombine-ant mouse TLR2-Fc protein at a fixed concentration (1 μg/ml, in 1% BSA-PBS) with an equal volume of CEP-PE vesicles at concentrations from 0 to 125 μM was added to each well, and the plate was allowed to incubate for 1 h at 37° C. DPPE vesicle solutions at 0, 50, 75 and 120 μM concentration, prepared by sonication, were used as parallel samples. After washing the plate with PBS containing 0.1% Tween-20 (PBST) buffer, 100 μL of 1:2,000 diluted goat anti-human Fc antibody in PBST buffer was added to each well. The plate was then incubated at room temperature for 1 h followed by washing with PBST buffer, and finally developed by adding 100 μL ABTS solution to each well. After an additional 30 min incubation, the absorbance for each well was read at 405 nm with a microplate reader (Bio-Rad, Hercules, Calif.).

### Results

### PLD from Streptomyces Chromofuscus Catalyzes Hydrolysis of CEP-PE to Generate CEP-ETN

The complexity of lipid side chains in naturally occurring EPs presents a challenging problem for CEP-EP detection by LC-MS because CEP derivatives are distributed into dozens of different diacyl, 1- alkyl-2-acyl and 1-alkenyl-2-acyl ethanolamine phosphoglycerides. However, the analysis can be simplified by enzymatic hydrolysis of the phospholipids. We exploited phospholipase D (PLD) from Streptomyces chromofuscus to release CEP-ethanolamine (CEP-ETN) from the polar head of CEP-EP derivatives. We anticipated that this method would eliminate the diversity inherent in naturally occurring EPs by converting all of the different CEPEPs into a single molecule, CEP-ETN.

To verify CEP-PEs are substrates of PLD, CEP-DPPE and d₄-CEP-PE standards were treated with PLD from Streptomyces chromofuscus. Then the generation of CEP-ETN and d₄-CEPETN was monitored by LC-MS/MS, respectively. LC-MS/MS data in FIG. 1 showed that, prominent levels of CEP-ETN and d₄-CEP-ETN peaks were observed in the hydrolyzed CEP-PE or d₄-CEP-PE samples, suggesting that these CEP-PEs are readily hydrolyzed by PLD (Streptomyces chromofuscus) to release CEP modified ethanolamine headgroups (FIGS. 1C and 1D).

### LC-MS/MS Demonstration that CEP-EPs Are Present in Human Plasma

Lipid extracts from 200 μL of AMD and normal human plasma samples were hydrolyzed by 280 units of PLD (Streptomyces chromofuscus). d₄-CEP-PE (20 ng, 0.023 nmol) was added to each sample as internal standard before PLD hydrolysis. Then the CEP ETN levels in the hydrolysis reaction product mixtures were determined by LC-MS/MS.

To minimize possible interference from variations in phospholipolysis efficiency and interference from the complex biological matrices that may vary among samples, a phospholipid internal standard was implemented for the LC-MS/MS analysis. A d₄-CEP-PE standard was prepared from L-a-phosphatidylcholine (egg PC) by an enzyme mediated transphosphatidylation. The d₄-CEP-PE standard obtained was a mixture containing deuterated CEP-PEs with various fatty acyl side chains, which strongly mimics the natural multiplicity of CEP-PE fatty acyl side chains in biological matrices. FIG. 1 shows the LC-MS/MS chromatograms from 20 ng of d₄-CEP-PE standard treated either with or without PLD hydrolysis. FIG. 1D suggests that hydrolyzed d₄-CEP-PE shows no interference from the MRM transition 184.3→124.2 found in the chromatograph of CEP-ETN. In FIG. 1B, although a trace amount of d4-CEP-ETN was present in the d₄-CEP-PE, its molar percentage was less than 7% or 1.6% in mass percentage. A correction for this trace impurity could be applied during data analysis (vide infra).

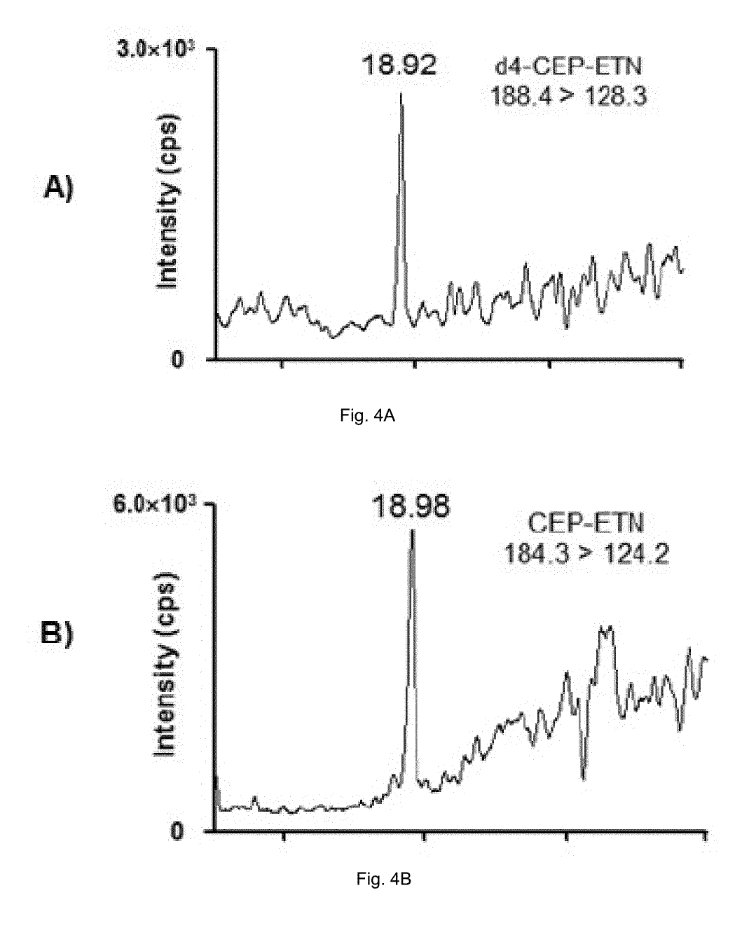

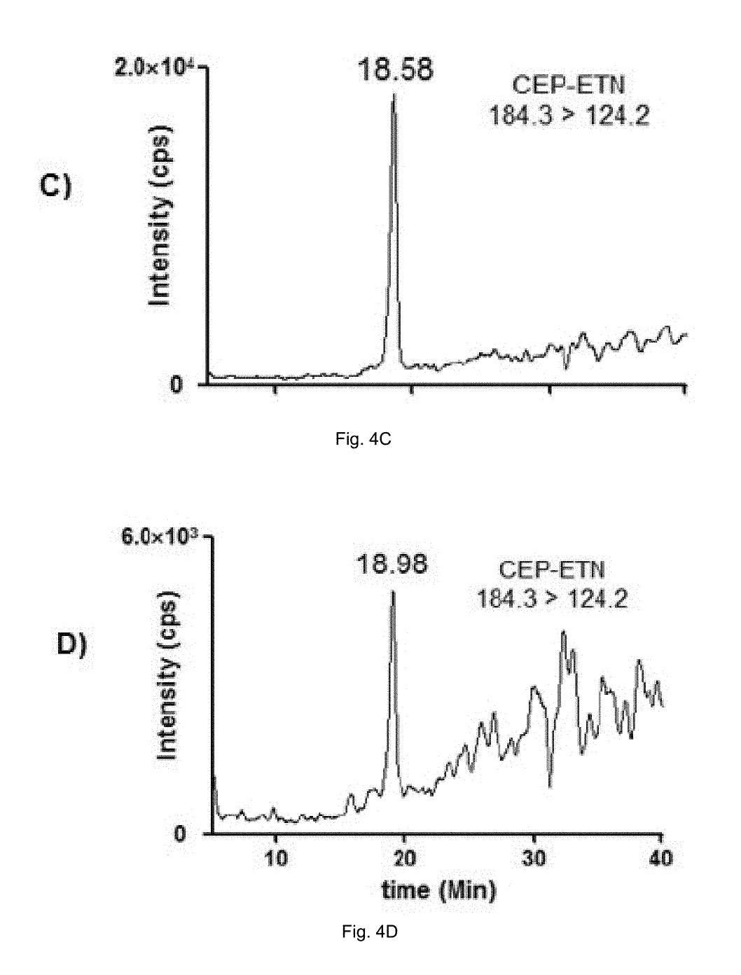

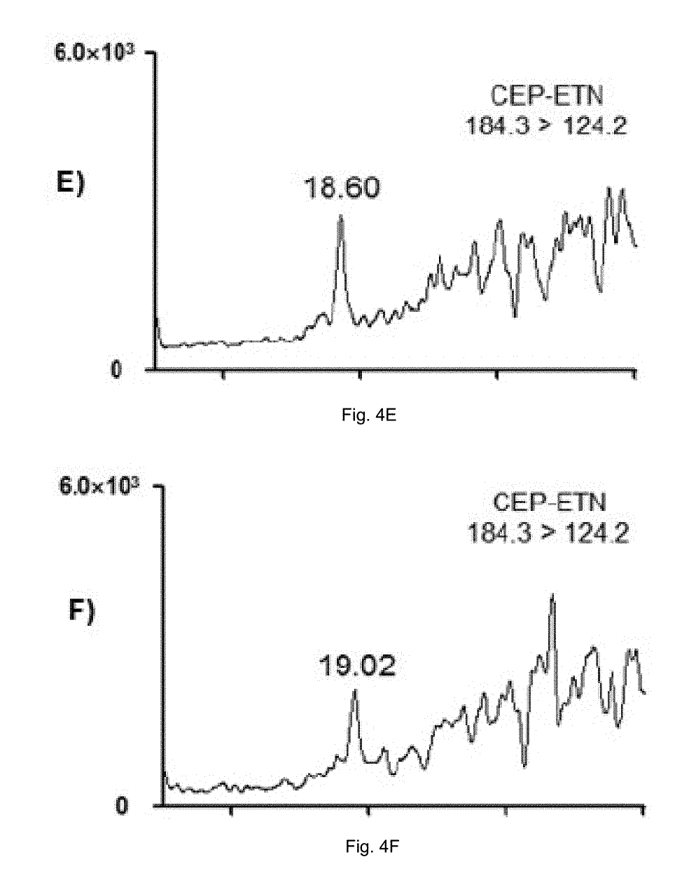

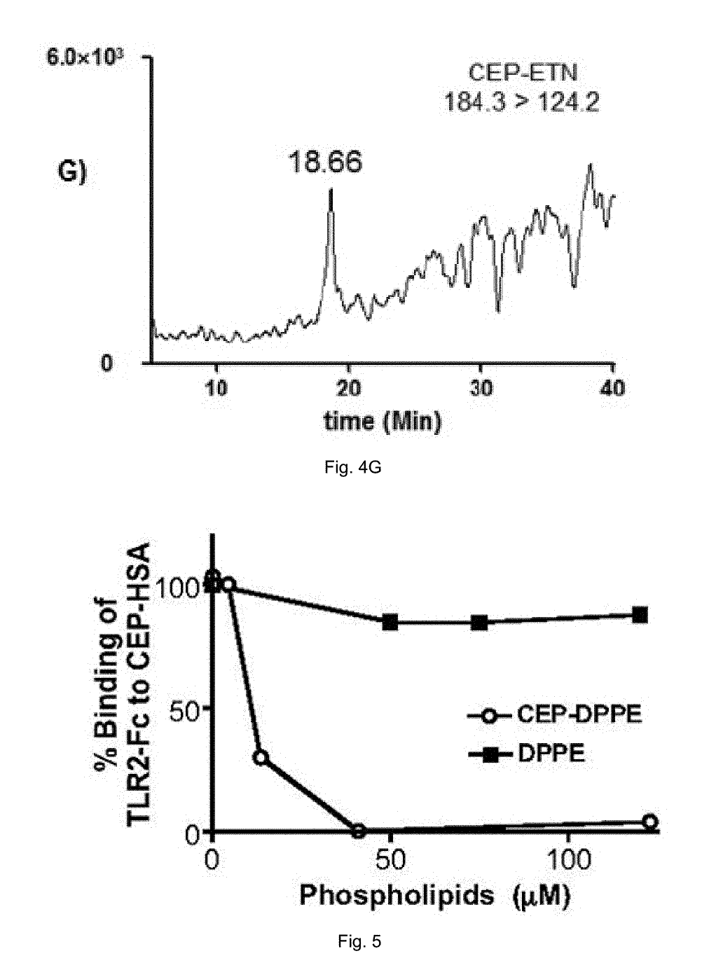

A calibration curve for CEP-ETN quantification was established with samples containing pure CEP-ETN in HBSS buffer in the concentration range from 2-30 nM. A fixed amount of internal standard, d₄-CEP-ETN (0.023 nmol), was added to 200 μL of each calibrator standard. The peak area ratio of CEP-ETN to d₄-CEPETN from the LC-MS/MS analysis was ploted against calibrator concentrations. For human plasma samples, the CEP-PE concentration was calculated as follows: [CEP-PE]=[Peak area ratio of (CEP-ETN)/(d4-CEPETN)×1.074-0.0272]/0.059 (nM) where 1.074 is the factor to correct interference caused by the trace of d4-CEP-ETN present in the d4-CEP-PE standard. The LC-MS/MS data demonstrate that CEP-EP derivatives are present in human blood plasma. Representative LC- MS/MS chromatograms of CEP-ETN detected in PLD hydrolyzed lipid extracts from human plasma samples are shown in FIG. 4.

### CEP-EP Levels are Elevated in AMD Plasma

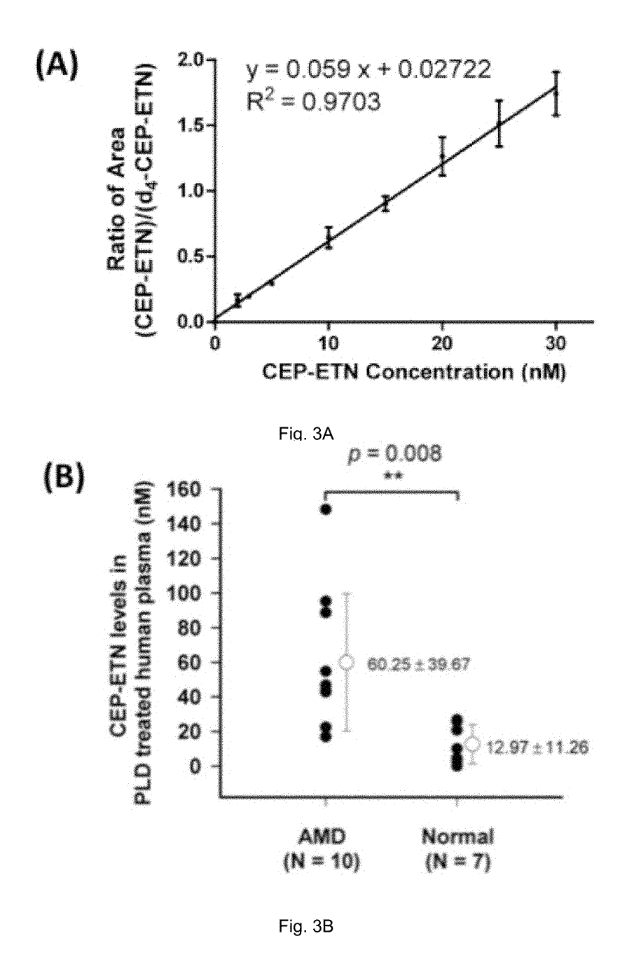

LCMS/MS analysis of human plasma samples from donors with AMD (n=10) and normal controls (n=7) indicates that, the CEP-EP derivative concentration in AMD samples is nearly 4.6-fold higher than its level in normal plasma samples (60±40 μmol/mL vs 13±11 pmol/mL; p<0.01), as shown in FIG. 3B. These results demonstrate that levels of CEP-EPs in vivo are strongly associated with age-related macular degeneration.

### CEP-DPPE Induces Tube Formation of Cultured HUVECs

Using pure CEP-DPPE prepared by unambiguous chemical synthesis, we conducted in vitro tube formation assays to evaluate the pro-angiogenic effect of CEP-PE derivatives toward human umbilical vein endothelial cells (HUVECs). Two experiments, with three replicates each, were performed by two independent individuals; one used PBS buffer as negative control and the other used DPPE as negative control. Both experiments indicate that CEP-DPPE induces tube formation by HUVECs (FIGS. 2A and 2B), suggesting that CEP-DPPE promotes angiogenesis, similar to VEGF, CEP-protein and CEP-peptide derivatives (FIG. 2A). Previously the mechanism of CEP-protein induced angiogenesis was shown to involve the TLR2 cell signaling pathway consequent to direct binding of CEP-protein to TLR2.7. The CEP moiety was found to be responsible for the binding. Thus, it seemed reasonable to postulate that CEP-PE would also bind to TLR2 and induce tube formation through TLR2 signaling. To test this hypothesis, we measured the binding affinity of CEP-DPPE vesicles to a recombinant mouse-TLR2-Fc fusion protein (mTLR2-Fc, R&D systems, Minneapolis, Minn.) in a competitive binding assay. CEPDPPE was shown to compete with CEP-HSA in binding with mTLR2-Fc protein, but DPPE does not inhibit the binding of CEPHSA with TLR2 (FIG. 5).

Direct evidence that CEP-PE induces angiogenesis through the activation of a TLR2 signaling pathway was then provided by the observation that oxPAPC, a TLR2/TLR4 inhibitor, inhibited (p<0.001 or p<0.02 versus control) the induction of HUVEC tube formation by CEP-DPPE (FIG. 2C). In addition, knockdown of TLR2 (kdn) by TLR2 siRNA also abolished (p<0.005 versus control or p<0.03) the ability of CEP-DPPE to induce tubeformation by HUVECs.

In summary, this Example indicates that a lipid oxidation product from docosahexaenoate containing phospholipids modifies EPs in vivo generating CEP-EPs. CEP-EPs are found in human plasma, and their levels are significantly elevated in plasma from donors with age-related macular degeneration. CEP-PE exhibits proangiogenic activity that is dependent on the TLR2 signaling pathway as found previously for CEP-protein and peptide derivatives.

### EXAMPLE 2

In this Example, we show that a sensitive quantitative simultaneous analysis of the various carboxyalkyl pyrrole derivatives of ethanolamine phospholipids can be achieved in a single LC-MS/MS experiment using isotope-labeled standards. The method is exemplified with an analysis of the levels of these pyrroles in blood from individuals with sickle cell disease (SCD). Elevated blood levels were found during routine clinical monitoring of SCD patients and lower levels in patients during hospitalization to treat a sickle cell crisis.

### Experimental Procedures

### Materials

PLD from Streptomyces sp. and Streptomyces chromofuscus were obtained from Enzo Life Sciences (Farmingdale, N.Y.). Hank's balanced salt solution (HBSS) buffer was purchased from Thermo Scientific (Waltham, Mass.). Ethanol-1,1,2,2-d4-amine (ETN-d4, 99 atom % D) was purchased from C/D/N Isotopes (Quebec, Canada). 6-[1,3]Dioxolan-2-yl-4-oxo-hexanoic acid-9H-fluoren-9-yl methyl ester, and 6-(1,3-dioxan-2-yl)-4-oxohexanoic acid were provided by Dr. Li Hong. 7-(1.3-Dioxan-2-yl)-5-oxoheptanoic acid and 11-(1,3-dioxan-2-yl)-9-oxoundecanoic acid were provided by Dr. Liang Xin. 4,7-Dioxoheptanoic acid 9H-fluoren-9-ylmethyl ester (DOHA-Fm, 2) was prepared as described previously. All other chemicals and reagents were obtained from Sigma-Aldrich unless specified.

### 7-Dioxoheptanoic acid 9H-fluoren-9-ylmethyl ester (DOHA-Fm)

Ester acetal (300 mg, 0.76 mmol) in 40 mL of AcOH/H2O (3:1, v/v) was stirred at 50° C. for 5 h under Ar protection. The reaction was monitored by TLC (hexane/ethylacetate/diethyl ether 2:1:1). After TLC analysis showed completion of the reaction, the solvents were evaporated under reduced pressure and the residue was purified by flash chromatography on a silica gel column (CH₂Cl₂, TLC: R_{f}=0.17) to give pure DOHA-Fm (223.3 mg, 0.66 mmol, 88%). ¹H NMR (400 MHz, CDCl₃) δ 9.79 (s, 1H), 7.77 (d, J=7.5 Hz, 2H), 7.59 (dd, J=7.5, 0.8 Hz, ²H), 7.41 (t, J=7.4 Hz, ²H), 7.32 (td, J=7.5, 1.2 Hz, ²H), 4.39 (d, J=7.1 Hz, ²H), 4.22 (t, J=7.1 Hz, ¹H), 2.79-2.69 (m, 8H).

### 3-(1-(2-Hydroxyethyl)-1H-pyrrol-2-yl)propanoic acid (CEP-ETN)

DOHA-Fm (92.5 mg, 0.27 mmol) reacted with ethanolamine (0.33 mmol) in 5.0 mL of mixture of MeOH/CH₂Cl₂ (1:1, v/v) for 7 h at room temperature under nitrogen protection to afford CEP-ETN-Fm ester. Then, 75 mL of DBU was added and the mixture was incubated overnight at room temperature to remove the Fm group. TLC was used to monitor reaction progress (CH₂Cl₂/CH₃OH 5:1). After TLC analysis showed completion of the reaction, the solvents were evaporated under reduced pressure and the residue was purified by flash chromatography on a silica gel column (CH₂Cl₂/CH₃OH 5:1, TLC: R_{f} =0.4) to give pure CEP-ETN (33.0 mg, 0.18 mmol, 66%). 1H NMR (400 MHz, CD₃OD) δ 6.65-6.61 (m, 1H), 5.98-5.94 (m, 1H), 5.82 (dd, J=3.0, 1.3 Hz, 1H), 3.95 (t, J=5.9 Hz, 2H), 3.74 (t, J=5.9 Hz, 2H), 2.87 (t, J=7.6 Hz, 2H), 2.62 (dd, J=8.4, 6.8 Hz, 2H). ESI-MS: m/z calcd for C9H14NO3 [M+H]⁺, 184.10, found 184.20; m/z calcd for C₉H₁₂NO₃ [M−H], 182.08, found 182.07.

### 3-(1-(2-Hydroxy-1,1,2,2-D,D,D,D-ethyl)-1H-pyrrol-2-yl)propanoic acid (CEP-ETN-d₄)

DOHA-Fm (48 mg, 0.14 mmol) was incubated with ethanol-1,1,2,2-d4-amine (0.17 mmol) in 3.0 mL of a mixture of MeOH/CH₂Cl₂ (1:1, v/v) for 6 h at room temperature under nitrogen protection to afford CEP-ETN-d4-Fm ester. Then, 32 mL of DBU was added and the mixture was incubated overnight at room temperature to remove the Fm group. TLC was used to monitor progress of the reaction (CH₂Cl₂/CH₃OH 5:1). After TLC analysis showed completion of the reaction, the solvents were evaporated under reduced pressure and the residue was purified by flash chromatography on a silica gel column (CH₂Cl₂/CH₃OH 5:1, TLC: R_{f}=0.4) to give pure CEP-ETN-d4 (16.8 mg, 0.09 mmol, 68%). ¹H NMR (400 MHz, CD₃OD) δ 6.63 (dd, J=2.8, 1.8 Hz, 1H), 5.97-5.93 (m, 1H), 5.84-5.80 (m, 1H), 2.91-2.84 (m, 2H), 2.62 (dd, J=8.4, 6.9 Hz, 2H). ESI-MS: m/z calcd for C₉H₁₀D₄NO₃ [M+H]⁺, 188.12, found 188.40; m/z calcd for C₉H₈D₄NO₃ [M−H], 186.11, found 186.08.

### 4,6-Dioxoheptanoic acid (DOHA)

Acetal (240 mg, 1.2 mmol) in a solution of AcOH/H20 (20 mL, 3:1, v/v) was stirred at 50° C. for 4 h under Ar protection. TLC (CH₂Cl₂/CH₃OH 10:1) was used to monitor progress of the reaction. Once the reaction was complete, the solvent was removed by rotary evaporation to give crude DOHA (7, 172.7 mg, 1.09 mmol, 91%), which was used without purification for pyrrole syntheses.

### 5,8-Dioxooctanoic acid (DOOA)

Acetal (115 mg, 0.5 mmol) in 20 mL of AcOH/H₂O (3:1, v/v) was stirred at 50° C. for 5 h under Ar protection. TLC (CH₂Cl₂/CH₃OH 10:1) was used to monitor progress of the reaction. Once the removal of the acetal was complete, the solvent was removed by rotary evaporation to give crude DOOA (80 mg, 0.46 mmol, 93%), which was used without purification for pyrrole syntheses.

### 9,12-Dioxododecanoic acid (DODA)

Acetal (114 mg, 0.40 mmol) in 20 mL of AcOH/H₂O (3:1, v/v) was stirred at 50° C. for 5 h under Ar protection. TLC (CH₂Cl₂/CH₃OH 10:1) was used to monitor progress of the reaction. Once the reaction was complete, the solvent was removed by rotary evaporation to give crude DODA (86 mg, 0.38 mmol, 95%), which was used without purification for pyrrole syntheses.

### 4-(1-(2-Hydroxyethyl)-1H-pyrrol-2-yl)butanoic acid (CPP-ETN)

The keto aldehyde (17.2 mg, 0.1 mmol) was allowed to react with ethanolamine (0.15 mmol) in 8 mL of MeOH for 6.5 h at room temperature under Ar protection. TLC was used to monitor the reaction progress (CH₂Cl₂/CH₃OH 5:1). After TLC analysis showed completion of the reaction, the solvents were evaporated under reduced pressure and the residue was purified by flash chromatography on a silica gel column (CH₂Cl₂/CH₃OH 5:1, TLC: R_{f}=0.45) to give pure CPP-ETN (14.2 mg, 0.072 mmol, 72%). ¹H NMR (400 MHz, CD₃OD) δ 6.65-6.60 (m, 1H), 5.96 (t, J=3.1 Hz, 1H), 5.82 (s, 1H), 3.94 (t, J=6.0 Hz, 2H), 3.73 (t, J=5.9 Hz, 2H), 2.62 (t, J=7.6 Hz, 2H), 2.36 (t, J=7.3 Hz, 2H), 1.96-1.84 (m, 2H). ESI-MS: m/z calcd for C₁₀H₁₆NO₃ [M+H]⁺, 198.11, found 198.20.

### 4-(1-(2-Hydroxyethyl-1,1,2,2-d4)-1H-pyrrol-2-yl)butanoic acid (CPP-ETN-d₄)

The keto aldehyde (25.8 mg, 0.15 mmol) was allowed to react with ethanol-1,1,2,2-d4-amine (0.225 mmol) in 15 mL of MeOH for 6 h at room temperature under Ar protection. TLC was used to monitor the process of the reaction (CH₂Cl₂/CH₃OH 5:1). After TLC analysis showed the completion of the reaction, the solvents were evaporated under reduced pressure and the residue was purified by flash chromatography on a silica gel column (CH₂Cl₂/CH₃OH 5:1, TLC: R_{f}=0.45) to give pure CPP-ETN-d₄ (21.2 mg, 0.105 mmol, 70%). ¹H NMR (400 MHz, CD₃OD) δ 6.62 (dd, J=2.8, 1.8 Hz, 1H), 5.97-5.94 (m, 1H), 5.83-5.79 (m, 1H), 2.65-2.59 (m, 2H), 2.35 (t, J=7.3 Hz, 2H), 1.93-1.85 (m, 2H). ESI-MS: m/z calcd for C10H12D4NO3 [M+H]⁺, 202.14, found 202.20; m/z calcd for C₁₀H₁₀D₄NO₃ [M−H]−, 200.12, found 200.20.

### 8-(1-(2-Hydroxyethyl)-1H-pyrrol-2-yl)octanoic acid (CHP-ETN)

The keto aldehyde (22.8 mg, 0.1 mmol) was allowed to react with ethanolamine (0.15 mmol) in 8 mL of MeOH for 8 h at room temperature under Ar protection. TLC was used to monitor progress of the reaction (CH₂Cl₂/CH₃OH 10:1). After TLC analysis showed the completion of the reaction, the solvents were evaporated under reduced pressure and the residue was purified by flash chromatography on a silica gel column (CH₂Cl₂/CH₃OH/AcOH 10:0.3:0.1, TLC: R_{f}=0.2) to give pure CHP-ETN (19.3 mg, 0.076 mmol, 76%). ¹H NMR (400 MHz, CD₃OD) δ 6.63-6.59 (m, 1H), 5.94 (t, J=3.1 Hz, 1H), 5.78 (s, 1H), 3.92 (t, J=6.0 Hz, 2H), 3.72 (t, J=6.0 Hz, 2H), 2.61-2.52 (m, 2H), 2.28 (t, J=7.4 Hz, 3H), 1.61 (d, J=4.6 Hz, 4H), 1.38 (s, 6H). ESI-MS: m/z calcd for C₁₄H₂₄NO₃ [M+H]⁺, 254.18, found 254.50.

### 4-(1-(2-Hydroxyethyl-1,1,2,2-d4)-1H-pyrrol-2-yl)butanoic acid (CHP-ETN-d₄)

The keto aldehyde (27.4 mg, 0.12 mmol) was allowed to react with ethanol-1,1,2,2-d4-amine (0.18 mmol) in 15 mL of MeOH for 7 h at room temperature under Ar protection. TLC was used to monitor the process of the reaction (CH₂Cl₂/CH₃OH 10:1). After TLC analysis showed the completion of the reaction, the solvents were evaporated under reduced pressure and the residue was purified by flash chromatography on a silica gel column (CH₂Cl₂/CH₃OH/AcOH 10:0.3:0.1, TLC: R_{f}=0.2) to give pure CHP-ETN-d4 (24.1 mg, 0.094 mmol, 78%). ¹H NMR (400 MHz, CD₃OD) δ 6.62-6.58 (m, 1H), 5.94 (t, J=3.1 Hz, 1H), 5.77 (s, 1H), 2.59-2.51 (m, 2H), 2.15 (d, J=7.5 Hz, 3H), 1.60 (s, 4H), 1.36 (d, J=12.6 Hz, 6H). ESI-MS: m/z calcd for C₁₄H₂OD₄NO₃ [M+H]⁺, 258.20, found 258.30; m/z calcd for C₁₄H₁₈D₄NO₃ [M−H]⁻, 256.19, found 256.27.

### CEP derivative of 1,2-dipalmitoyl-sn-glycero-3-phosphoethanolamine (CEP-DPPE)

CHCl₃/CH₃OH (6 mL, 2:1, v/v) was added into a vial containing DOHA (23.7 mg, 0.15 mmol) and DPPE (69.2mg, 0.1 mmol). Then triethylamine (TEA, 40 μL, 0.4 mmol) was added and the mixture was allowed to react overnight at room temperature under argon. TLC (CH₂Cl₂/CH₃OH 10:1) was used to monitor progress of the reaction. After TLC analysis showed completion of the reaction, the solvents were evaporated under reduced pressure and the residue was purified by flash chromatography on a silica gel column (CH₂Cl₂/MeOH 10:1, R_{f}=0.28) to give CEP-DPPE (12, 56.9 mg, 0.07 mmol, 70%). ¹H NMR (400 MHz, CDCl₃/CD₃OD, 1/1): δ 6.61 (s, 1H), 6.00-5.95 (m, 1H), 5.82 (d, J=3.4 Hz, 1H), 5.13 (s, 1H), 4.30 (dd, J=12.0, 2.9 Hz, 1H), 4.05 (dt, J=11.5, 5.9 Hz, 5H), 3.71 (d, J=4.0 Hz, 2H), 2.88 (t, J=7.5 Hz, 2H), 2.68-2.61 (m, 2H), 2.30-2.24 (m, 6H), 1.57 (s, 4H), 1.24 (s, 47H), 0.85 (d, J=6.8 Hz, 6H). ESI-MS: m/z calcd for C₄₄H₇₉NO₁₀P⁻ [M−H]⁻, 812.54, found 812.40; calcd for C₄₄H₈₁NO₁₀P⁺ [M+H]⁺ 814.56, found 814.13.

### CPP derivative of 1,2-dipalmitoyl-sn-glycero-3-phosphoethanolamine (CPP-DPPE)

DOOA (13.1 mg, 0.076 mmol) was allowed to react with DPPE (34.0 mg, 0.049 mmol) in CHCl₃/MeOH (6 mL, 2:1, v/v) containing TEA (30 μL, 0.296 mmol) for 7 hours at room temperature under argon. After TLC analysis showed completion of the reaction, the solvents were evaporated under reduced pressure and the residue was purified by silica gel flash chromatography (CHCl₃/MeOH 3:1, R_{f}=0.2) to give CPP-DPPE (26.4 mg, 0.032 mmol, 65%). ¹H NMR (400 MHz, CDCl₃/CD₃OD, 1:1): δ=6.53 (s, 1H), 5.93 (t, J=3.2 Hz, 1H), 5.75(s, 1H), 5.07 (m, 1H), 4.25 (dd, J=2.1, 12.0 Hz, 1H), 4.07 (m, 1H), 3.98 (m, 4H), 3.63(m, 2H), 2.57 (t, J=8.0 Hz, 2H), 2.29 (t, J=6.4 Hz, 2H), 2.22 (m, 4H), 1.85 (m, 2H), 1.51 (m, 2H), 1.18 (48H), 0.80 (t, J=2.4 Hz, 6H). ESI-MS: m/z calcd for C₄₅H₈₁NO₁₀P⁻ [M−H]⁻, 826.56, found 826.40; calcd for C₄₅H₈₃NO₁₀P⁺[M+H]⁺828.56, found 828.40.

### CHP derivative of 1,2-dipalmitoyl-sn-glycero-3-phosphoethanolamine (CHP-DPPE)

DODA (32.2 mg, 0.141 mmol) was allowed to react with DPPE (95.4 mg, 0.137 mmol) in CHCl₃/MeOH (12 mL, 2:1, v/v) containing TEA (60 μL, 0.592 mmol) for 7 hours at room temperature under argon. After TLC analysis showed completion of the reaction, the solvents were evaporated under reduced pressure and the residue was purified by silica gel flash chromatography (CHCl₃/MeOH 5:1, Rf=0.19) to give CHP-DPPE (82.3 mg, 0.093 mmol, 67%). ¹H NMR (400 MHz, CDCl₃/CD₃OD 2:1): δ=6.54 (s, 1H), 5.92 (t, J=3.2 Hz, 1H), 5.74 (s, 1H), 5.07 (m, 1H), 4.23 (dd, J=2.8, 12.0 Hz, 1H), 4.00 (dd, J=7.2, 12.0 Hz, 1H), 3.94 (m, 4H), 3.65 (m, 2H), 2.46 (t, J=7.6 Hz, 2H), 2.22 (m, 6H), 1.5 (m, 8H), 1.18 (54H), 0.80 (t, J=6.8 Hz, 6H). ESI-MS: m/z calcd for C₄₉H₈₉NO₁₀P⁻ [M−H]⁻, 882.62, found 882.47; calcd for C₄₉H₉₁NO₁₀P⁺[M+H]⁺884.63, found 884.33.

### Nonane-1,4-diol

γ-Nonalactone (1 mL, 976 mg, 6.25 mmol) was added to 20 mL of tetrahydrofuran (THF) in a flask and the solution was cooled with a Dry Ice-acetone bath. Then 13.0 mL of 1 M of lithium aluminum hydride in THF (13 mmol) was added dropwise over 10 min while cooling continued. After the flask was allowed to warm to room temperature, the reaction mixture was stirred for an additional 3 h. After cooling in an ice bath, ice-cold saturated ammonium chloride (5.0 mL) was added until the evolution of hydrogen stopped. Then the ice bath was removed and 40 mL of potassium sodium tartate tetrahydrate was added. The resulting mixture was allowed to stir slowly for 15 min. Then the mixture was transferred to separatory funnel and extracted with ethyl acetate and water. The upper organic phase was collected and dried over magnesium sulfate. The solvents were evaporated under reduced pressure to give crude nonane-1,4-diol (702 mg, 4.38 mmol, 70%), which was used for the without further purification for the preparation of 4-oxononanal. 1H NMR (400 MHz, CDCl₃) 6 3.72-3.55 (m, 3H), 1.74-1.58 (m, 3H), 1.49-1.22 (m, 9H), 0.88 (q, J=7.0 Hz, 3H).

### 4-Oxononanal

Oxalyl dichloride (2.5 mL, 3.69 g, 29 mmol) in a three-necked flask was cooled with a dry ice acetone bath. Then dimethyl sulfoxide (DMSO, 5 mL, 5.5 g, 70.4 mmol) was added and the resulting mixture was allowed to stir for 45 min while cooling continued. Diol (1.16 g, 7.2 mmol) was added slowly to the mixture, which was then allowed to stir for another 70 min with continued cooling in a dry ice acetone bath. TEA (8 mL, 57 mmol) was then added slowly and the mixture was stirred for another 2 min. Then the mixture was allowed to warm to room temperature. The reaction was quenched by the addition of a mixture of water and dichloromethane. The mixture was extracted three times with dichloromethane and the lower organic phase was collected and dried over anhydrous sodium sulfate. The solvents were evaporated under reduced pressure and the residue was purified by flash chromatography on a silica gel column (15% ethyl acetate in hexanes, R_{f}=0.2) to give 4-oxononanal (968 mg, 6.2 mmol, 86%). ¹H NMR (400 MHz, CDCl3) δ 9.77 (d, J=0.6 Hz, 1H), 2.74-2.68 (m, 4H), 2.46-2.40 (m, 2H), 1.56 (ddd, J=14.8, 10.8, 7.5 Hz, 2H), 1.31-1.21 (m, 4H), 0.86 (dd, J=8.9, 5.2 Hz, 3H).

### 2-(2-Pentyl-1H-pyrrol-1-yl)ethan-1-ol (PP-ETN)

4-Oxononanal (50 mg, 0.32 mmol) and ethanolamine (0.48 mmol) were incubated in 50.0 mL of MeOH/CH₂Cl₂ (1:1, v/v) overnight at room temperature under argon. TLC was used to monitor progress of the reaction (30% ethyl acetate in hexanes). After TLC analysis showed completion of the reaction, the solvents were evaporated under reduced pressure and the residue was purified by flash chromatography on a silica gel column (15% ethyl acetate in hexanes, TLC: R_{f}=0.15) to give PP-ETN (41.7 mg, 0.23 mmol, 73%). ¹H NMR (400 MHz, CD3OD) δ 6.61 (dd, J=2.8, 1.8 Hz, 1H), 5.96-5.93 (m, 1H), 5.78 (ddt, J=3.4, 1.7, 0.8 Hz, 1H), 3.92 (t, J=6.1 Hz, 2H), 3.72 (t, J=6.0 Hz, 2H), 2.59-2.52 (m, 2H), 1.66-1.57 (m, 2H), 1.40-1.35 (m, 4H), 0.95-0.91 (m, 3H). ESI-MS: m/z calcd for C₁₁H₂₀NO [M+H]⁺, 182.15, found 182.40.

### 2-(2-Pentyl-1H-pyrrol-1-yl)ethan-1,1,2,2-d4-1-ol (PP-ETN-d₄)

4-Oxononanal (70 mg, 0.45 mmol) and ethanol-1,1,2,2-d4-amine (0.68 mmol) in 50.0 mL of a mixture of MeOH/CH₂Cl₂ (1:1, v/v) were incubated overnight at room temperature under argon. TLC was used to monitor progress of the reaction (15% ethyl acetate in hexanes). After TLC analysis showed the completion of the reaction, the solvents were evaporated under reduced pressure and the residue was purified by flash chromatography on a silica gel column (15% ethyl acetate in hexanes, TLC: R_{f}=0.15) to give pure PPETN- d4 (59 mg, 0.32 mmol, 72%). ¹H NMR (400 MHz, CD₃OD) 6 6.60 (dd, J=2.8, 1.8 Hz, 1H), 5.95 (dd, J=3.4, 2.8 Hz, 1H), 5.78 (ddt, J=3.4, 1.7, 0.8 Hz, 1H), 2.58-2.53 (m, 2H), 1.61 (tdd, J=7.1, 5.7, 3.2 Hz, 2H), 1.38 (ddd, J=4.4, 2.1, 0.9 Hz, 4H), 0.95-0.91 (m, 3H). ESI-MS: m/z calcd for C₁₁H₁₆D₄NO [M+H]+, 186.18, found 186.40.

### General Methods

Proton magnetic resonance (1H NMR) spectra and carbon magnetic resonance (¹³C NMR) spectra were recorded on a Varian Inova AS400 spectrometer operating at 400 MHz and 100 MHz, respectively. Proton chemical shifts are reported as parts per million (ppm) on the 6 scale relative to CDCl₃ (δ 7.26) or CD₃OD (δ 3.31). ¹H NMR spectral data are tabulated in terms of multiplicity of proton absorption (s, singlet; d, doublet; dd, doublet of doublet; t, triplet; q, quartet; m, multiplet; br, broad), coupling constants (Hz), number of protons. Carbon chemical shifts are reported relative to CDCl₃ (δ 77.0) or CD₃OD (δ 49.0). Flash chromatography was performed with ACS grade solvents from Fisher Scientific (Hanover Park, Ill.). R_{f} values are quoted for TLC plates of thickness 0.25 mm from Whatman (Florham Park, N.J.). The plates were visualized with iodine, dinitrophenylhydrazine or phosphomolybdic acid reagents. For all reactions performed in an inert atmosphere, argon was used unless otherwise specified.

### Extraction of Lipids from Blood Plasma

Clinically documented sickle cell disease blood was obtained from anonymized patients in the clinic and from hospitalized patients at the MetroHealth Medical Center. For this pilot study, blood was drawn during hospitalization and not at admission. Human blood samples from SCD patients were collected in 7 ml tubes (lavender top) containing EDTA (10.5 mg). After incubating the tubes at room temperature for 30 min, the upper yellow plasma layer was transferred into a 15 mL Falcon tube (Fisher Cat. #14-959-70C). BHT (5 μL/mL) and 10 μL/mL of protease inhibitor cocktail (Sigma, Cat.# P8340) were added and mixed gently 5-6 times by inversion of the tube. Remaining blood cells were removed by centrifugation at 2500 rpm (1300×g) for 20-30 min at 4° C. Two mL of the supernatant was aliquoted into 8 vials (250 μL/vial) (Fisher Scientific Cat. No 02-681-343) with screw caps (Fisher Scientific Cat. No 02-681-358). The vials were flushed with argon, sealed with screw caps, and then quench-frozen in liquid nitrogen for 1 min and then stored at −80° C. Phospholipids were extracted from the plasma by a modified Bligh and Dyer method. In brief, 750 μL of chloroform/methanol (1:2, v/v) containing 1 mM BHT was added to the vial containing 200 μL of plasma. The mixture was vortexed vigorously. Then 250 μL of chloroform was added to the mixture, which was vortexed vigorously again. Aqueous sodium chloride solution (250 μL of 1.5%) was added and the resulting mixture was vortexed vigorously followed by centrifuging for 10 min at 3,000 RPM to give a three-phase system (aqueous top, protein disk, organic bottom). The organic phase was withdrawn very carefully with a Pasteur pipette. The lipids were dried under a stream of dry nitrogen. The residue was stored under argon at −80° C. until analysis.

### Phospholipase D (Streptomyces chromofuscus) Mediated Hydrolysis of Lipids

The isolated phospholipids were hydrolyzed with PLD (Streptomyces chromofuscus) by the method described previously for hydrolysis of CEP-modified EPs. Briefly, lipids were resuspended in 50 μL of methanol followed by the addition of 450 μL of HBSS buffer supplemented with 5 mM CaCl₂ and 0.1 mM EDTA. Then the mixture was incubated at 37° C. for 30 minutes and then sonicated for 10 minutes. Finally, small unilammelar vesicles (SUVs) were generated by passing the cloudy lipid mixture through a 0.1 μm polycarbonate filter (17 times) for extrusion using an Avanti Mini-Extruder Set (Avanti Polar Lipids, Inc., Alabaster, Ala.). Then, 280 units of phospholipase D (Streptomyces chromofuscus) were added into the SUV solution and the resulting mixture was shaken under argon protection at 37° C. overnight. Then the solvent was evaporated under a stream of dry nitrogen. The residue was stored at −80° C. under Ar and dissolved in 100 μL of methanol and 20 μL of this solution was injected when doing LC-MS/MS analysis.

### High Performance Liquid Chromatography/Mass Spectrometry

LC-ESI/MS/MS analysis of CAPETN and PP-ETN derivatives was performed on a Quattro Ultima triple-quadrupole mass spectrometer (Micromass, Wythenshawe, UK) equipped with a Waters Alliance 2690 HPLC system with an autoinjector (Waters, Milford, Mass.). The chromatographic separation was achieved using a Luna C18 column (150 2.0 mm i.d. 5 μm, Phenomenex). The source and desolvation temperature was maintained at 120° C. and 250° C., respectively. The drying gas (N2) and cone flow gas were kept at ca. 650 L/h and 65 L/h, respectively. The multiplier was set at an absolute value of 600. LC-MS/MS analysis was performed in the positive ion mode and the total run time was 45 min. The mobile phase consisted of solvent A (HPLC grade water containing 0.1% formic acid) and solvent B (HPLC graded methanol containing 0.1% formic acid). The HPLC gradient steps were set as follows: 0-5 min, isocratic at 5% solvent B; 5-25 min, linear gradient from 5 to 100% solvent B; 25-35 min, isocratic at 100% solvent B; 35-45 min, isocratic at 5% solvent B. The flow rate employed was 200 μL/min.

Optimized parameters for detection of CAP-ETN and PP-ETN derivatives were determined with authentic samples. MS scans at m/z 30-330 were obtained for standard compounds. For multiple reaction monitoring experiments, argon was used as collision gas at a pressure of 5 psi. The optimum collision energy and other parameters were determined for each individual analyte.

### Statistical Analysis

Statistical analyses were performed by using Student's t test. A P value <0.05 is considered as statistically significant. Representative p-values in figures include “NS”-not significant, “*” p<0.05, “**” p<0.002, “***” p<0.0001. Data are presented as mean±SD.

### Results

### Simultaneous Quantitative Analysis of CAP-EPs and PP-EPs in Biological Samples

Total phospholipids isolated from biological samples by a modified Bligh & Dyer method, were prepared for phospholipolysis by conversion into small unilamellar vesicles (SUVs) by extrusion. After addition of internal standards CAP-ETN-d₄ and PP-ETN-d₄, phospholipase D from Streptomyces chromofuscus was added to catalyze the release CAP-ETN and PP-ETN. The resulting product mixture was then analyzed by injection into the LC-MS/MS. In a control experiment, quantitative release of CEP-ETN from CEP-DPPE was observed after incubation for 1 h (data not shown). Nevertheless, for phospholipids extracted from biological samples, incubation with PLD was always allowed to proceed overnight.

### Syntheses of CAP and PP Derivatives of ETN and DPPE

All CAP derivatives of ETN and deuterated ETN as well as of DPPEs were prepared by unambiguous chemical syntheses. The nondeuterated authentic CEP-ETN and deuterated authentic internal standard CEP-ETN-d4 were synthesized. Briefly, the propylene acetal group of 6-[1,3]dioxolan-2-yl-4-oxohexanoic acid-9H-fluoren-9-yl-methyl ester prepared was removed by treatment with aqueous acetic acid to give the 9H-fluoren-9-ylmethyl ester of 4,7-dioxoheptanoic acid (DOHA-Fm). Finally, CEP-ETN and CEP-ETN-d₄ were generated by the reaction of 2 with ETN or ethanol-1,1,2,2-d₄-amine (ETN-d4) through a Paal-Knorr reaction followed by the removal of the Fm ester by 1,8-diazabicyclo[5.4.0]undec-7-ene.

For other authentic CAP-ETN derivatives (CPP-ETN and CHP-ETN) and deuterated CAP-ETN derivatives (CPP-ETN-d₄ and CHP-ETN-d₄) as well as authentic CAP-DPPE derivatives (CEP-DPPE, CPP-DPPE and CPP-DPPE), there was no need to protect the carboxyl group to obtain a high level of modification. In brief, the propylene acetal group of the dioxolane-protected carboxylic acid was removed by treatment with aqueous acetic acid to give the corresponding γ-keto aldehyde carboxylic acids that delivered carboxyalkylpyrrole derivatives by Paal- Knorr synthesis.

For the synthesis of non-deuterated authentic PP-ETN and deuterated authentic internal standard PPETN- D4, the Paal-Knorr synthesis was successfully applied to the generation of PP derivatives from 4-oxononanal. 4-Oxononanal was prepared by a method published elsewhere with minor modification. Briefly, the commercially available starting material γ-nonalactone was first reduced by LiAlH4 to nonane-1,4-diol, which was then converted to 4-oxononanal through Swern oxidation.

### Test the Hypothesis that Pyrrole Modified EPs are Substrates for PLD from Streptomyces chromofuscus

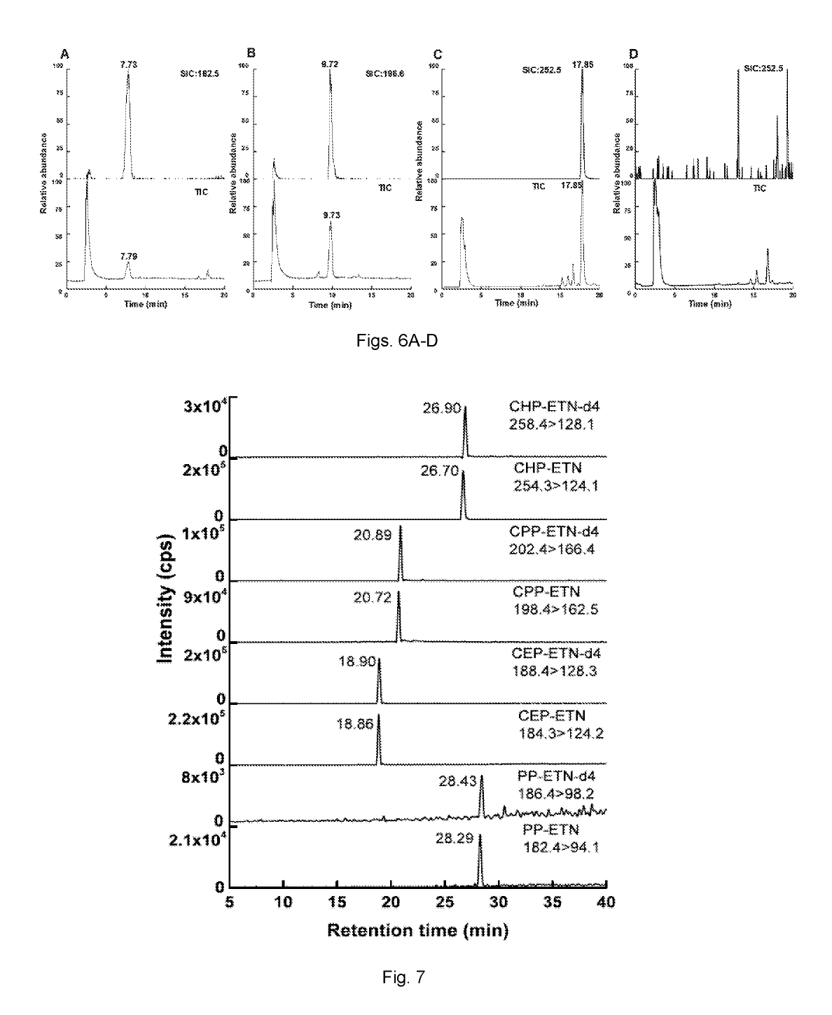

Although PLD is an efficient lipase of levuglandin and isolevuglandin derivatives of EPs (also referred to as γ-KA-EPs) and CEP-EPs, whether PLD can hydrolyze other pyrrole modified EPs was not known. Therefore, the ability of PLD (Streptomyces chromofuscus) to catalyze the hydrolysis of pyrrole derivatives of EP was evaluated by treatment authentic CAP-DPPE with PLD from Streptomyces chromofuscus followed by analysis by LC-MS in the negative ion mode. As shown in FIG. 6A-C, significant peaks were observed in the selected ion chromatograms (SICs) corresponding to CAP-ETNs after hydrolysis of CAP-DPPEs catalyzed by PLD from Streptomyces chromofuscus, confirming that CAP-DPPEs are substrates for PLD (Streptomyces chromofuscus). To verify that the CAP-ETN was produced by PLD-catalyzed hydrolysis of CAP-DPPE, the hydrolysis product from CHP-DPPE without PLD (Streptomyces chromofuscus) treatment was also studied by LCMS as a representative. As shown in FIG. 6D, no peak corresponding to CHP-ETN was observed in the SIC of the reaction product mixture from hydrolysis of CHP-DPPE in the absence of PLD.

### LC-MS/MS Analysis of Authentic CAP and PP Derivatives of ETN and Deuterated ETN

For the analysis of CAP and PP derivatives of EPs in biological samples by reverse phase HPLC/ESI/MS/MS using multiple reaction monitoring (MRM), a MRM method was developed based on the collision induced dissociation (CID) spectra of [M+H]⁺ of the parent ion for authentic CAP and PP derivatives of ETN and deuterated ETN in the positive ion mode. MRM of the transitions 184.3→124.2, 188.4→128.2, 198.4→162.5, 202.4→166.4, 254.3→124.1, 258.4→128.1, 182.4→94.1 and 186.4→98.2 were then used to analyze CEP-ETN, CEP-ETN-d₄, CPP-ETN, CPP-ETNd₄, CHP-ETN, CHP-ETN-d₄, PP-ETN and PP-ETN-d₄, respectively. Therefore, these eight MRM transitions were set to monitor the LC-MS/MS of a mixture of all eight authentic standards. As shown in FIG. 7, there are strong signals for each channel in the MRM. Signals for the CEP, CPP, CHP and PP derivatives are well separated by LC. Consequently, this LCMS/ MS method can be used for the simultaneous analysis of all three CAP-ETNs and PP-ETN in biological samples.

### LC-MS/MS Demonstration that CAP and PP Derivatives of EPs are Present in Human Plasma

Sickle cell disease (SCD) is a class of hemoglobinopathy in humans clinically characterized by chronic hemolysis, inflammation and vaso-occlusion, causing a decreased quality of life and life expectancy. Chronically elevated oxidative stress is an important feature of SCD and might play a significant role in the pathophysiology of a cascade of SCD-related debilitating conditions. Despite the mounting evidence of oxidative stress in SCD, there is still no practical biomarker for determining the degree of oxidative stress and disease severity in SCD. Since CAP and PP derivatives have been measured immunologically in human blood plasma, we anticipated that CAP and PP derivatives of EPs exist in human plasma and might serve as biomarkers indicative of the disease state in SCD.

Based on the workflow developed for detection of CAP and PP derivatives of EPs in biological samples, their presence in blood plasma samples from routine clinical monitoring of SCD patients and hospitalized patients during a sickle cell crisis was examined. In brief, phospholipids were first extracted from 200 μL of human plasma samples from hospitalized and clinic patients with SCD by a modified Bligh & Dyer method. Then lipid extracts were prepared as LMV followed by the addition of a fixed amount of each of the internal standards, CAP-ETN-d4 and PP-ETNd4 (0.01 nmol) and the mixtures were then hydrolyzed by 280 units of PLD (Streptomyces chromofuscus). The amounts of CAP-ETN and PP-ETN released from blood lipids in the hydrolysis reaction product mixtures were determined by the LC-MS/MS methods described above. To determine the linearity and sensitivity of the assay for measurement of CAP-ETN and PP-ETN, calibration curves were constructed with authentic CAP-ETN and PP-ETN in 200 μL of HBSS buffer in the concentration range from 4 to 80 nM spiked with a fixed amount of each of internal standards, CAPETN- d4 and PP-ETN-d4 (0.01 nmol). The peak area ratio of CAP-ETN or PP-ETN to CAP-ETN-d4 or PP-ETN-d4 from the LC-MS/MS analysis was plotted against calibrator concentrations to generate the corresponding curves and equations as shown in FIG. S5A-D. These equations were then used to calculate the measured amount of CAP-ETN and PP-ETN in the hydrolysis reaction product mixtures from human blood plasma samples. For human plasma samples, the CAP or PP-EP concentration was calculated as follows:

[CAP-EP or PP-EP]=[Area(CAP-EP or PP-ETN)/Area(CAP-EP-d4 or PP-ETN-d4)−Intercept]×1000/2Slope (nM)

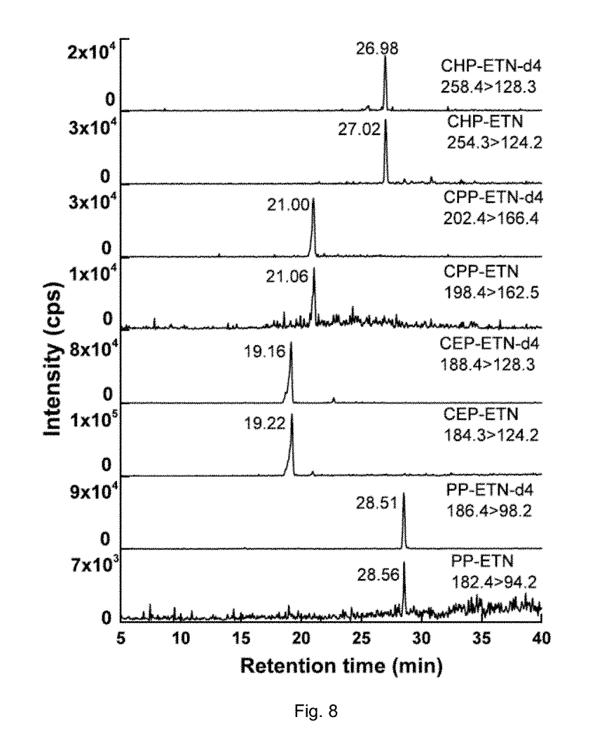

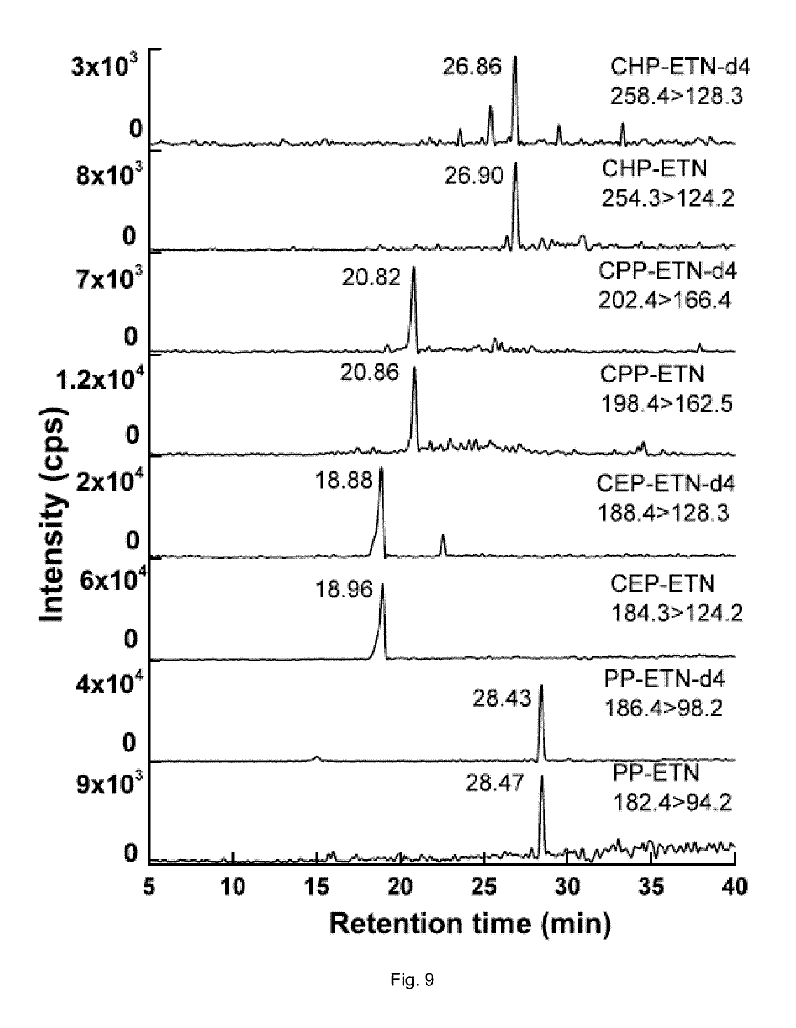

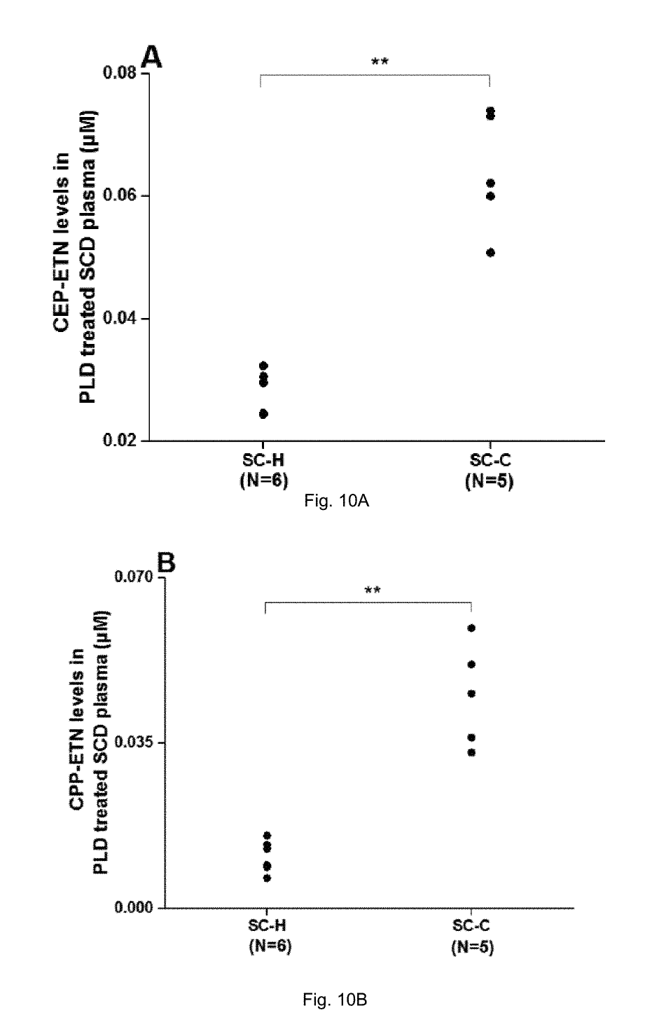

The LC-MS/MS analysis of lipid extracts after treatment with PLD revealed the presence of all CAP EP and PP-EP derivatives in human blood plasma. Representative LC-MS/MS chromatograms of CAPETN and PP-ETN detected in PLD-hydrolyzed lipid extracts from human blood plasma samples from hospitalized and clinic patients with SCD are shown in FIGS. 8 and 9.

### Blood Levels of CEP-EP, CPP-EP and PP-EP are Massively Elevated in SCD

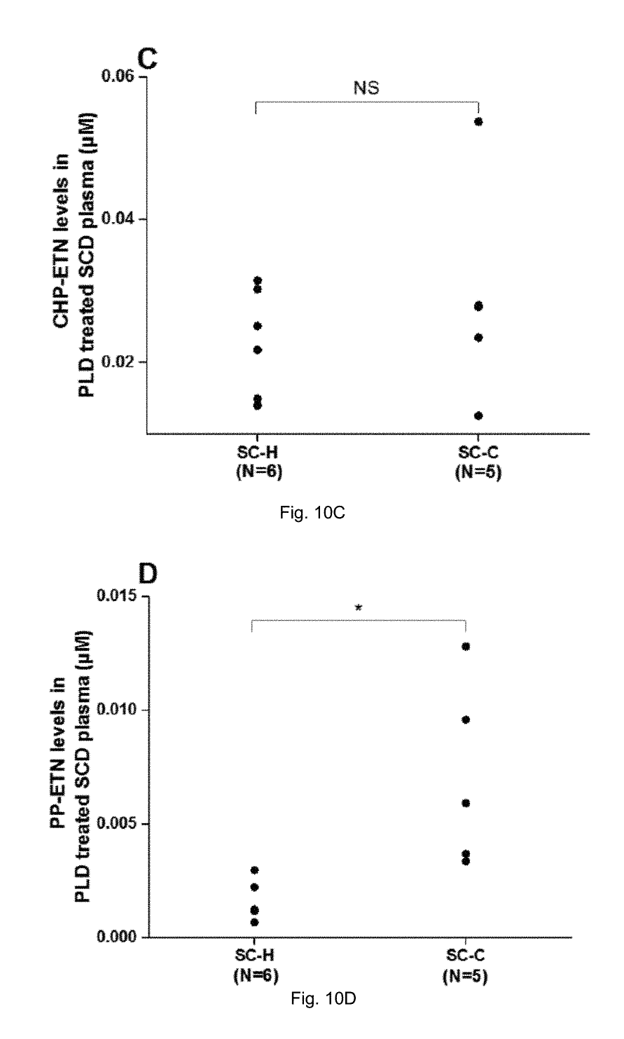

We applied an LC-MS/MS analysis to simultaneously determine CAP-EP and PP-EP levels in blood plasma from routine clinical monitoring of SCD patients (SC-C, n=5) and from SCD patients hospitalized to treat a sickle cell crisis (SC-H, n=6). The results are summarized in Table 1. Uniformly elevated blood levels of CEP-EP (63.9±9.7 nM) are found during routine clinical monitoring of SCD patients. Although mean CEP-EP levels are similarly elevated in blood from AMD patients (56.3±37.1 nM) compared to individuals with no disease (12.1±10.5 nM), there is much greater variation in the levels detected in AMD compared to SCD. One factor that could account for this variability is the localization in the eye of the oxidative injury in AMD. To be detected in the blood, CEP-EPs or their precursors must cross the blood-retina barrier. There may be significant differences in the diseaserelated permeability of that barrier. As shown in FIG. 10A and Table 1, two fold lower levels (P<0.002) of CEP-EPs (27.6±3.6 nM, n=5) were detected in plasma from SCD patients hospitalized to treat a sickle cell crisis. It is important to emphasize that all the hospitalized patients had their blood drawn during hospitalization and not at admission and had received multiple blood transfusions, significant amounts of intravenous fluids for rehydration, and strong narcotics for pain control. This quickly attenuated their pain crisis presumably limiting further oxidative damage. The dilution of their plasma volume by these therapeutic approaches most likely influenced our results, giving lower marker values than in non-hospitalized patients not in a pain crisis. Further studies will be needed to obtain a blood sample from each sickle cell disease patient in a pain crisis at admission just prior to any therapeutic intervention. This shows that therapeutic measures have some efficacy in lowering CEP-PE levels, although mean levels remain higher than those (12.1±10.5 nM) detected in blood from healthy controls in a previous study of CEPEP levels in blood from AMD patients.

TABLE 1

| | | | | CHP-EP |
|---|---|---|---|---|
| | PP-EP (nM) | CEP-EP (nM) | CPP-EP (nM) | (nM) |
| | (mean ± SD) | (mean ± SD) | (mean ± SD) | (mean ± SD) |
| SC-H | 1.48 ± 0.92 | 27.6 ± 3.6 | 10.9 ± 3.4 | 229. ± 7.5 |
| (N = 6) | | | | |
| SC-C | 7.06 ± 4.05 | 63.9 ± 9.7 | 45.1 ± 10.9 | 29.1 ± 15.1 |
| (N = 5) | | | | |
| AMD | | 56.3 ± 37.1 | | |
| (N = 10) | | | | |
| Normal | | 12.1 ± 10.5 | | |
| (N = 7) | | | | |

Similarly, as shown in FIG. 10B and Table 1, plasma levels of CPP-EPs from SCD clinic patients were four-fold higher than those of SCD patients hospitalized to treat a sickle cell crisis (45.1±10.9 versus 10.9±3.4 nM; p<0.002). Although the mean levels of CHP-EPs (shown in FIG. 5C and Table 1) detected in plasma from hospitalized SCD patients (22.9±7.5 nM, n=5) was lower than in plasma from SCD clinic patients (29.1±15.1 nM, n=6 patients) the difference is not statistically significantly (NS). As shown in FIG. 5D and Table 1, PP-EP concentrations in plasma from SCD patients is nearly 4.8-fold higher than its level in plasma samples from SCD patients hospitalized to treat a sickle cell crisis (7.06±4.05 vs 1.48±0.92 nM; p<0.05). Nevertheless, levels of CAP-PEs are 4 to 9 fold higher than those of PP-EP. This pilot study demonstrated that massively elevated levels of CAP-EPs are present in blood from SCD patients detected during routine clinical monitoring.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A method of detecting carboxyalkylpyrrole ethanolamine phospholipids (CAP-EPs) or pentylpyrrole ethanolamine phospholipids (CAP-EPs) in a bodily sample from a subject, the method comprising:
obtaining a bodily sample from a subject suspected of including carboxyalkylpyrrole ethanolamine phospholipids (CAP-EPs) or pentylpyrrole ethanolamine phospholipids (PP-EPs);
extracting carboxyalkylpyrrole ethanolamine phospholipids or pentylpyrrole ethanolamine phospholipids from the bodily sample;
hydrolyzing carboxyalkylpyrrole or pentylpyrrole ethanolamine phospholipids from the extracted with a phospholipase D to form carboxyalkylpyrrole ethanolamine (CAP-ETN) and pentylpyrrole ethanolamine (PP-ETN) derivatives; and
determining the amount of carboxyalkylpyrrole ethanolamine (CAP-ETN) and pentylpyrrole ethanolamine (PP-ETN)by mass spectrometry, wherein the amount of carboxyalkylpyrrole ethanolamine (CAP-ETN) and pentylpyrrole ethanolamine (PP-ETN) is determinative of the level of carboxyalkylpyrrole or pentylpyrrole ethanolamine phospholipids in the sample.

2. The method of claim 1, wherein the carboxyalkylpyrrole ethanolamine phospholipids (CAP-EPs) or pentylpyrrole ethanolamine phospholipids (PP-EPs) are extracted from the bodily sample in the presence of at least one of a chelating agent or antioxidant to inhibit oxidation of the bodily sample.

3. The method of claim 2, wherein the chelating agent or antioxidant includes at least one of ethylenediaminetetraacetic acid (EDTA) or butylated hydroxytoluene (BHT).

4. The method of claim 1, the bodily sample includes at least one of blood, plasma, or sera.

5. The method of claim 1, wherein the subject has or at risk of age-related macular degeneration (AMD) and the level of the one or more carboxyalkylpyrrole ethanolamine phospholipids (CAP-EPs) or pentylpyrrole ethanolamine phospholipids (PP-EPs) measured is compared to a control value; and characterizing the subject as at greater risk of developing AMD if the level of the one or more carboxyalkylpyrrole ethanolamine phospholipids (CAP-EPs) or pentylpyrrole ethanolamine phospholipids (PP-EPs) measured is greater that than the control value or characterizing the subject as at lesser risk of developing AMD if the level of one or more carboxyalkylpyrrole ethanolamine phospholipids (CAP-EPs) or pentylpyrrole ethanolamine phospholipids (PP-EPs)measured is not greater than the control value.

6. The method of claim 5, wherein the AMD is advanced AMD and the control value is a control value for mild to moderate AMD.

7. The method of claim 5, wherein the AMD is mild to moderate AMD and the control value is a control value for non-AMD.

8. A method of determining a subject has or at is at risk of age-related macular degeneration (AMD), the method comprising:
obtaining a bodily sample from a subject suspected of including carboxyalkylpyrrole ethanolamine phospholipids (CAP-EPs) or pentylpyrrole ethanolamine phospholipids (PP-EPs);
extracting carboxyalkylpyrrole ethanolamine phospholipids or pentylpyrrole ethanolamine phospholipids from the bodily sample;
hydrolyzing carboxyalkylpyrrole or pentylpyrrole ethanolamine phospholipids from the extracted with a phospholipase D to form carboxyalkylpyrrole ethanolamine (CAP-ETN) and pentylpyrrole ethanolamine (PP-ETN) derivatives;
determining the amount of carboxyalkylpyrrole ethanolamine (CAP-ETN) and pentylpyrrole ethanolamine (PP-ETN)by mass spectrometry, wherein the amount of carboxyalkylpyrrole ethanolamine (CAP-ETN) and pentylpyrrole ethanolamine (PP-ETN) is determinative of the level of carboxyalkylpyrrole or pentylpyrrole ethanolamine phospholipids in the sample;
comparing the level of the one or more carboxyalkylpyrrole ethanolamine phospholipids (CAP-EPs) or pentylpyrrole ethanolamine phospholipids (PP-EPs) measured to a control value; and
characterizing the subject as at greater risk of developing AMD if the level of the one or more carboxyalkylpyrrole ethanolamine phospholipids (CAP-EPs) or pentylpyrrole ethanolamine phospholipids (PP-EPs) measured is greater that than the control value or characterizing the subject as at lesser risk of developing AMD if the level of one or more carboxyalkylpyrrole ethanolamine phospholipids (CAP-EPs) or pentylpyrrole ethanolamine phospholipids (PP-EPs)measured is not greater than the control value.

9. The method of claim 8, wherein the carboxyalkylpyrrole ethanolamine phospholipids (CAP-EPs) or pentylpyrrole ethanolamine phospholipids (PP-EPs) are extracted from the bodily sample in the presence of at least one of a chelating agent or antioxidant to inhibit oxidation of the bodily sample.

10. The method of claim 9, wherein the chelating agent or antioxidant includes at least one of ethylenediaminetetraacetic acid (EDTA) or butylated hydroxytoluene (BHT).

11. The method of claim 8, the bodily sample includes at least one of blood, plasma, or sera.

12. The method of claim 8, wherein the AMD is advanced AMD and the control value is a control value for mild to moderate AMD.

13. The method of claim 8, wherein the AMD is mild to moderate AMD and the control value is a control value for non-AMD.
